(19) United States
(12) Patent Application Publication
(10) Pub. No.: US 2025/0346579 A1
(43) Pub. Date: Nov. 13, 2025

(54) PYRIDAZINONE COMPOUND AND PREPARATION METHOD THEREFOR, PHARMACEUTICAL COMPOSITION THEREOF, AND APPLICATION THEREOF

(71) Applicant: CHINA PHARMACEUTICAL UNIVERSITY, Jiangsu (CN)

(72) Inventors: Yungen XU, Jiangsu (CN); Haiping HAO, Jiangsu (CN); Hongfeng GU, Jiangsu (CN); Wenxin YAN, Jiangsu (CN); Yi ZOU, Jiangsu (CN); Yong WANG, Jiangsu (CN); Hong WANG, Jiangsu (CN); Qihua ZHU, Jiangsu (CN); Wenbo XU, Jiangsu (CN); Jieping YANG, Jiangsu (CN); Yupei SU, Jiangsu (CN); Beibei LIU, Jiangsu (CN)

(73) Assignee: CHINA PHARMACEUTICAL UNIVERSITY, Jiangsu (CN)

(21) Appl. No.: 18/721,199

(22) PCT Filed: Dec. 22, 2022
(86) PCT No.: PCT/CN2022/141022

§ 371 Date: Nov. 25, 2024

(30) Foreign Application Priority Data
Dec. 23, 2021 (CN) 202111589174.2

(51) Int. Cl. C07D 403/14; A61K 31/501; A61K 31/506; C07D 405/14

(52) U.S. Cl. CPC C07D 403/14; A61K 31/501; A61K 31/506; C07D 405/14

(57) ABSTRACT

Disclosed in the present invention are a pyridazinone compound and a preparation method therefor, a pharmaceutical composition thereof, and an application thereof. The pyridazinone compound has a structure as shown in formula (I), and contains an isomer thereof, a pharmaceutically acceptable salt thereof, or a mixture of the isomer and the pharmaceutically acceptable salt. The pyridazinone compound has excellent pharmacokinetic properties in vivo, can also promote the release of immune factors, and in particular, also has excellent antitumor activity in vivo. In addition, the preparation method for the pyridazinone compound is simple and easy.

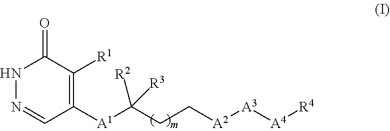

### TECHNICAL FIELD

The present invention relates to a pyridazinone compound and a preparation method therefor, a pharmaceutical composition thereof, and an application thereof, and particularly relates to a pyridazinone compound with anti-tumor activity and a preparation method therefor, a pharmaceutical composition thereof, and an application thereof.

### BACKGROUND

Malignant tumors are the leading cause of death affecting human health, and seriously endanger human life. In 2020, China reported 4.57 million new cancer cases and 3 million cancer-related deaths, accounting for 23.7% of new cases worldwide. For decades, surgery, radiotherapy, chemotherapy, targeted therapy, and the like, have significantly improved outcomes for cancer patients. However, these therapies have their own limitations. Both chemotherapy and targeted therapy are difficult to eradicate tumor cells in the body, and are prone to inducing drug resistance, which seriously affects treatment efficacy and prognosis.

Studies indicated that most members of the PARP family in the human body exhibited mono-ADP-ribosyltransferase activity. The monoPARP protein family is closely associated with the development of cancer, inflammation, and neurodegenerative diseases. PARP7, a member of the monoPARP protein family, is a novel negative regulator of nucleic acid sensors in cells and is overexpressed in tumors. Since cancer cells can use PARP7 to inhibit interferon signals and make them hide from the immune system, making many cancer cells reliant on PARP7 for survival. Studies revealed that inhibiting PARP7 could restore interferon signaling in cells, thereby reactivating both innate and adaptive immunity and inhibiting the growth of cancer cells. In cancer models such as lung cancer and colorectal cancer, PARP7 inhibitors have demonstrated sustained inhibition of tumor growth.

At present, no PARP7 inhibitors have been approved for marketing. RBN-2397 developed by Ribon Therapeutics is the first compound with strong inhibitory activity and selectivity against PARP7. In the xenograft tumor model of NCI-H1373 cells (human lung cancer adenocarcinoma cells), RBN-2397 exhibits significant anti-tumor effects at a dose of >30 mg/kg when being administered orally once a day, and is dose-dependent. RBN-2397 is currently being tested in Phase I clinical study (NCT04053673). However, due to its high in vivo clearance rate, RBN-2397 has low drug exposure and oral bioavailability in vivo. Results of in vivo animal efficacy studies indicated that administration of RBN-2397 alone is unlikely to produce significant anti-tumor effects, and it must be combined with a CYP450 inhibitor to reduce its clearance rate and achieve good anti-tumor effects.

### SUMMARY

An objective of the present invention: in view of the defects of poor pharmacokinetics, difficult single-drug function, difficulty in exerting effects as a single drug, and the like of the existing compounds, the present invention aims to provide a pyridazinone compound excellent in-vivo and in-vitro antitumor activity and pharmacokinetic properties, as well as a preparation method therefor, a pharmaceutical composition thereof, and an application thereof.

Technical solution: as a first aspect of the present invention, the pyridazinone compound provided in the present invention has a structure as shown in Formula (I), and contains an isomer thereof, a pharmaceutically acceptable salt thereof, or a mixture of the isomer and the pharmaceutically acceptable salt.

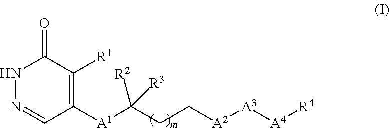

in the formula:
m is selected from 0, 1, 2 or 3;
R¹ is selected from hydrogen, halogen, cyano, trifluoromethyl, C₁-C₆ alkyl, C₁-C₆ alkoxy, methylthio, methanesulfonyl, and carbamoyl;
R² or R³ is independently selected from hydrogen, C₁-C₆ alkyl, substituted C₃-C₆ cycloalkyl or heterocycloalkyl, cyano, or trifluoromethyl; or R² and R³ together with carbon atoms to which they are connected form C₃-C₆cycloalkyl; a substituent of the C₃-C₆ cycloalkyl is selected from hydrogen, methyl, trifluoromethyl, 2,2-difluoroethyl, methoxy, halogen, cyano, amino, methylamino, dimethylamino, diethylamino, acetyl amino, acetoxy, methanesulfonyl, carboxyl or methoxycarbonyl, and the substituent is one or more;
R⁴ is selected from substituted aryl, substituted heteroaryl or substituted 1,3-benzodioxanyl, the heteroaryl or the 1,3-benzodioxanyl is selected from hydrogen, halogen, cyano, trifluoromethyl, 2,2-difluoroethyl, C₁-C₆ alkyl, C₁-C₆ alkoxy, C₃-C₆ cycloalkyl, aryl, hydroxy, methoxy, amino, methylamino, dimethylamino, acetyl amino, carboxyl, methanesulfonyl, methoxycarbonyl, or nitro, and a substituent is one or more;
A¹ is selected from —NH—, —O—, —S—,

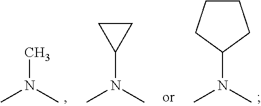

A² is selected from

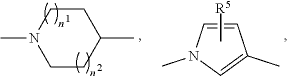

—NH— or —N(CH₃)—, where n¹ and n² are independently selected from 0, 1 or 2;
R⁵ is selected from hydrogen, halogen, methyl, trifluoromethyl, cyano, hydroxy, methoxy, amino, methylamino, dimethylamino, diethylamino or acetyl amino; and R⁵ is one or more;
A³ is selected from

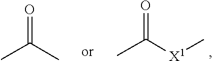

where X¹ represents —O—, —NH— or

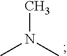

and
A⁴ is selected from

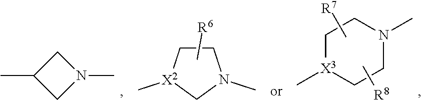

where X² and X³ are independently selected from N or CH, R⁶, R⁷ or R⁸ is independently selected from hydrogen, methyl, trifluoromethyl, cyano, hydroxy, methoxy, amino, methylamino, dimethylamino, acetyl amino, carboxyl or methoxycarbonyl.

Preferably, in the structure of the pyridazinone compound:
m is selected from 0, 1 or 2;
R¹ is selected from halogen, cyano or trifluoromethyl;
R² or R³ is independently selected from hydrogen, methyl or trifluoromethyl; and when
R² and R³ are different, carbon atoms connected to R² and R³ are in a racemic configuration, an R configuration, or an S configuration;
R⁴ is selected from

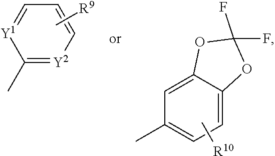

where Y¹ or Y² is independently selected from CH or N, R⁹ and R¹⁰ are independently selected from trifluoromethyl, methyl, fluorine, chlorine, bromine, cyano, methoxy, methanesulfonyl,2,2-difluoroethyl or 4-trifluoromethyl, and R⁹ or R¹⁰ is one or more;
A¹ is selected from —NH—;
A² is selected from

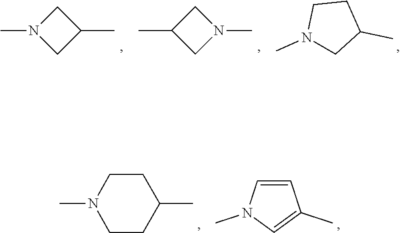

—NH— or —N(CH₃)—;
A³ is selected from

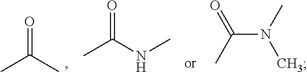

and
A⁴ is selected from

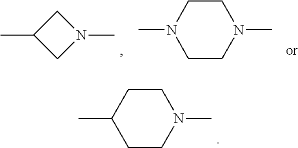

Further preferably, in the structure of the pyridazinone compound:
m is 0 or 2.

Further preferably, in the structure of the pyridazinone compound:
R¹ is trifluoromethyl; R² is hydrogen; R³ is hydrogen or methyl; and when R³ is methyl, a carbon atom connected to R³ is the S configuration.

Further preferably, in the structure of the pyridazinone compound:
A² is selected from

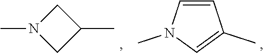

or —NH—; A³ is

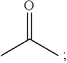

and A⁴ is

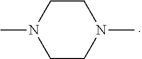

Further preferably, in the structure of the pyridazinone compound:
R⁴ is selected from

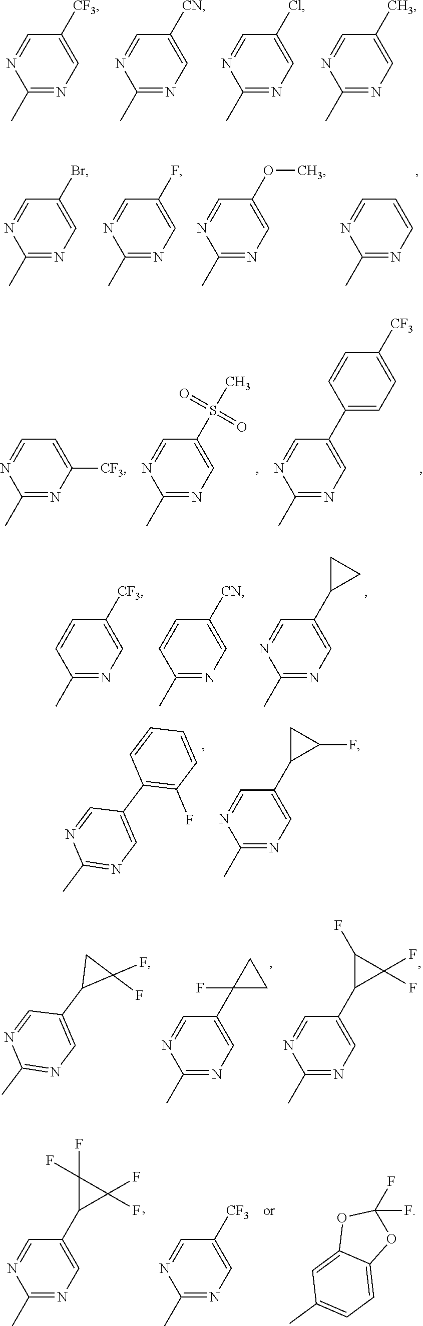

More specifically, the pyridazinone compound is any of the following compounds:

| Compound | | |
|---|---|---|
| No. | Compound naming | Compound structure |
| I-A-1 | (S)-4-(trifluoromethyl)-5-((1-(3-(4-(5- | |
| | (trifluoromethyl)pyrimidin-2- | |
| | yl)piperazine-1-carbonyl)azetidin-1- | |
| | yl)propan-2-yl)amino)pyridazin-3(2H)- | |
| | one | |
| I-A-2 | (S)-4-(trifluoromethyl)-5-((1-(3-(4-(5- (4-(trifluoromethyl)phenyl)pyrimidin- 2-yl)piperazine-1-carbonyl)azetidin-1- yl)propan-2-yl)amino)pyridazin-3(2H)- one | 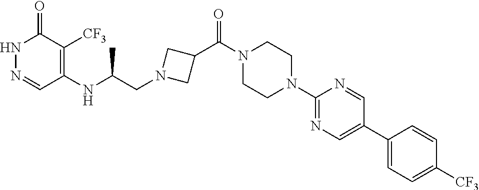 [SMILES: C[C@@H](CN1CC(C(=O)N2CCN(c3ncc(-c4ccc(C(F)(F)F)cc4)cn3)CC2)C1)Nc1cn[nH]c(=O)c1C(F)(F)F] |
| I-A-3 | (S)-2-(4-(1-(2-((6-oxo-5- (trifluoromethyl)-1,6-dihydropyridazin- 4-yl)amino)propyl)azetidine-3- carbonyl)piperazin-1-yl)pyrimidine-5- carbonitrile | 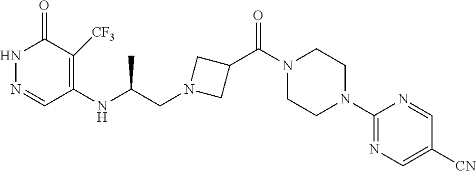 [SMILES: C[C@@H](CN1CC(C(=O)N2CCN(c3ncc(C#N)cn3)CC2)C1)Nc1cn[nH]c(=O)c1C(F)(F)F] |
| I-A-4 | (S)-5-((1-(3-(4-(5- (methylsulfonyl)pyrimidin-2- yl)piperazine-1-carbonyl)azetidin-1- yl)propan-2-yl)amino)-4- (trifluoromethyl)pyridazin-3(2H)-one | 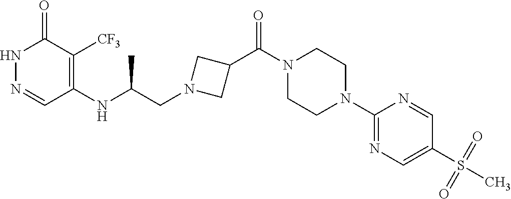 [SMILES: C[C@@H](CN1CC(C(=O)N2CCN(c3ncc(S(C)(=O)=O)cn3)CC2)C1)Nc1cn[nH]c(=O)c1C(F)(F)F] |
| I-A-5 | (S)-5-((1-(3-(4-(5-chloropyrimidin-2- yl)piperazine-1-carbonyl)azetidin-1- yl)propan-2-yl)amino)-4- (trifluoromethyl)pyridazin-3(2H)-one | 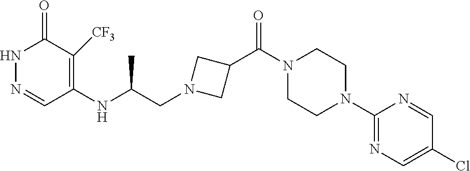 [SMILES: C[C@@H](CN1CC(C(=O)N2CCN(c3ncc(Cl)cn3)CC2)C1)Nc1cn[nH]c(=O)c1C(F)(F)F] |
| I-A-6 | (S)-5-((1-(3-(4-(5-methylpyrimidin-2- yl)piperazine-1-carbonyl)azetidin-1- yl)propan-2-yl)amino)-4- (trifluoromethyl)pyridazin-3(2H)-one | 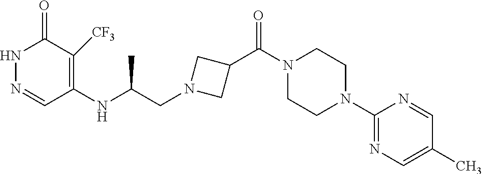 [SMILES: Cc1cnc(N2CCN(C(=O)C3CN(C[C@H](C)Nc4cn[nH]c(=O)c4C(F)(F)F)C3)CC2)nc1] |
| I-A-7 | (S)-5-((1-(3-(4-(5-bromopyrimidin-2- yl)piperazine-1-carbonyl)azetidin-1- yl)propan-2-yl)amino)-4- (trifluoromethyl)pyridazin-3(2H)-one | 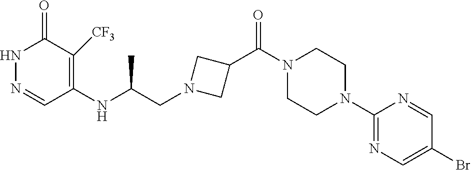 [SMILES: C[C@@H](CN1CC(C(=O)N2CCN(c3ncc(Br)cn3)CC2)C1)Nc1cn[nH]c(=O)c1C(F)(F)F] |
| I-A-8 | (S)-5-((1-(3-(4-(5-fluoropyrimidin-2- yl)piperazine-1-carbonyl)azetidin-1- yl)propan-2-yl)amino)-4- (trifluoromethyl)pyridazin-3(2H)-one | 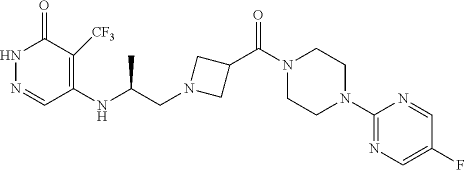 [SMILES: C[C@@H](CN1CC(C(=O)N2CCN(c3ncc(F)cn3)CC2)C1)Nc1cn[nH]c(=O)c1C(F)(F)F] |
| I-A-9 | (S)-5-((1-(3-(4-(5-methoxypyrimidin-2- yl)piperazine-1-carbonyl)azetidin-1- yl)propan-2-yl)amino)-4- (trifluoromethyl)pyridazin-3(2H)-one | 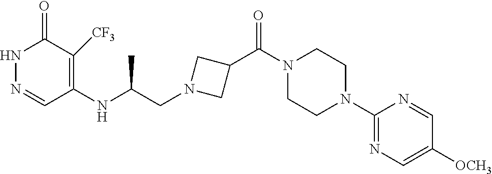 [SMILES: COc1cnc(N2CCN(C(=O)C3CN(C[C@H](C)Nc4cn[nH]c(=O)c4C(F)(F)F)C3)CC2)nc1] |
| I-A-10 | (S)-4-(trifluoromethyl)-5-((1-(3-(4-(5- (trifluoromethyl)pyridin-2- yl)piperazine-1-carbonyl)azetidin-1- yl)propan-2-yl)amino)pyridazin-3(2H)- one | 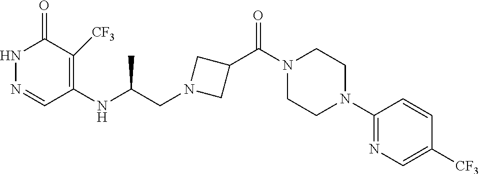 [SMILES: C[C@@H](CN1CC(C(=O)N2CCN(c3ccc(C(F)(F)F)cn3)CC2)C1)Nc1cn[nH]c(=O)c1C(F)(F)F] |
| I-A-11 | (S)-4-(trifluoromethyl)-5-((1-(3-(4-(4- trifluoromethyl)phenyl)piperazine-1- carbonyl)azetidin-1-yl)propan-2- yl)amino)pyridazin-3(2H)-one | 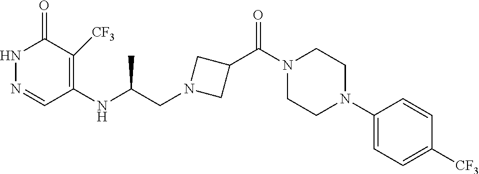 [SMILES: C[C@@H](CN1CC(C(=O)N2CCN(c3ccc(C(F)(F)F)cc3)CC2)C1)Nc1cn[nH]c(=O)c1C(F)(F)F] |
| I-A-12 | (S)-5-((1-(3-(4-dibenzo[b,d]furan-4- yl)piperazine-1-carbonyl)azetidin-1- yl)propan-2-yl)amino)-4- (trifluoromethyl)pyridazin-3(2H)-one | 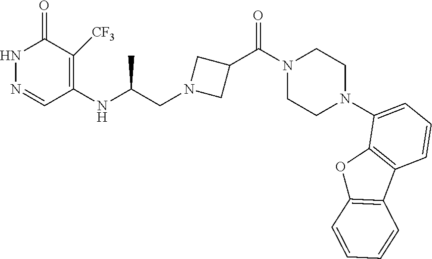 [SMILES: C[C@@H](CN1CC(C(=O)N2CCN(c3cccc4c3oc3ccccc34)CC2)C1)Nc1cn[nH]c(=O)c1C(F)(F)F] |
| I-A-13 | (S)-4-(trifluoromethyl)-5-((1-(3-(4-(4- (trifluoromethyl)pyrimidin-2- yl)piperazine-1-carbonyl)azetidin-1- yl)propan-2-yl)amino)pyridazin-3(2H)- one | 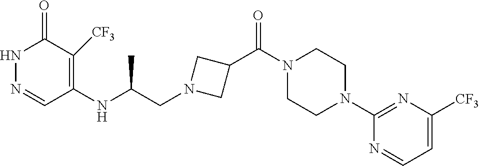 [SMILES: C[C@@H](CN1CC(C(=O)N2CCN(c3nccc(C(F)(F)F)n3)CC2)C1)Nc1cn[nH]c(=O)c1C(F)(F)F] |
| I-A-14 | (S)-5-((1-(3-(4-(pyrimidin-2- yl)piperazine-1-carbonyl)azetidin-1- yl)propan-2-yl)amino)-4- (trifluoromethyl)pyridazin-3(2H)-one | 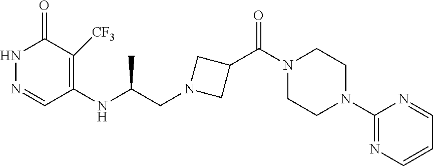 [SMILES: C[C@@H](CN1CC(C(=O)N2CCN(c3ncccn3)CC2)C1)Nc1cn[nH]c(=O)c1C(F)(F)F] |
| I-A-15 | (S)-1-(2-((6-oxo-5-(trifluoromethyl)- 1,6-dihydropyridazin-4- yl)amino)propyl)-N-(1-(5- (trifluoromethyl)pyrimidin-2- yl)azetidin-3-yl)azetidine-3- carboxamide | 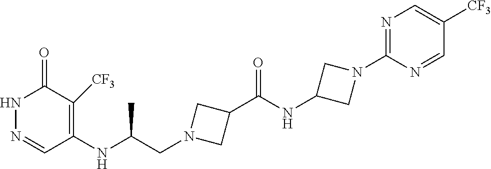 [SMILES: C[C@@H](CN1CC(C(=O)NC2CN(c3ncc(C(F)(F)F)cn3)C2)C1)Nc1cn[nH]c(=O)c1C(F)(F)F] |
| I-A-16 | (S)-N-methyl-1-(2-((6-oxo-5- (trifluoromethyl)-1,6-dihydropyridazin- 4-yl)amino)propyl)-N-(1-(5- (trifluoromethyl)pyrimidin-2- yl)azetidin-3-yl)azetidine-3- carboxamide | 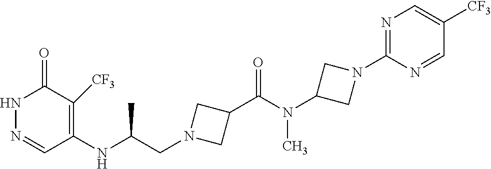 [SMILES: C[C@@H](CN1CC(C(=O)N(C)C2CN(c3ncc(C(F)(F)F)cn3)C2)C1)Nc1cn[nH]c(=O)c1C(F)(F)F] |
| I-A-17 | 5-(((2S)-1-(3-(2,6-dimethyl-4-(5- (trifluoromethyl)pyrimidin-2- yl)piperazine-1-carbonyl)azetidin-1- yl)propan-2-yl)amino)-4- (trifluoromethyl)pyridazin-3(2H)-one | 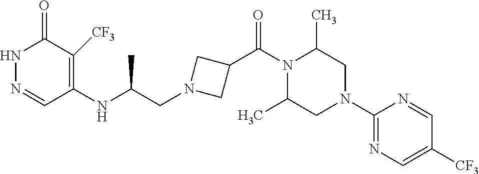 [SMILES: CC1CN(c2ncc(C(F)(F)F)cn2)CC(C)N1C(=O)C1CN(C[C@H](C)Nc2cn[nH]c(=O)c2C(F)(F)F)C1] |
| I-A-18 | 4-(trifluoromethyl)-5-(((2S)-1-(3-(4-(5- (trifluoromethyl)pyrimidin-2- yl)piperazine-1-carbonyl)pyrrolidin-1- yl)propan-2-yl)amino)pyridazin-3(2H)- one | 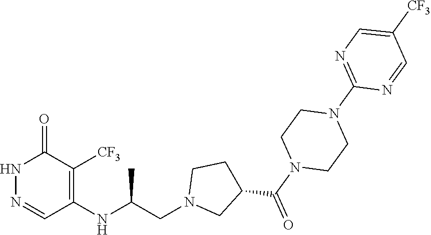 [SMILES: C[C@@H](CN1CC[C@H](C(=O)N2CCN(c3ncc(C(F)(F)F)cn3)CC2)C1)Nc1cn[nH]c(=O)c1C(F)(F)F] |
| I-A-19 | (S)-4-(trifluoromethyl)-5-((1-(4-(4-(5- (trifluoromethyl)pyrimidin-2- yl)piperazine-1-carbonyl)piperidin-1- yl)propan-2-yl)amino)pyridazin-3(2H)- one | 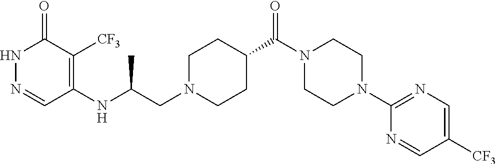 |
| I-A-20 | 4-(trifluoromethyl)-5-((2-(3-(4-(5- (trifluoromethyl)pyrimidin-2- yl)piperazine-1-carbonyl)azetidin-1- yl)ethyl)amino)pyridazin-3(2H)-one | 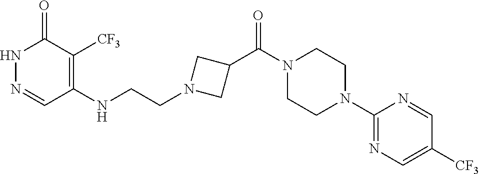 [SMILES: O=C(C1CN(CCNc2cn[nH]c(=O)c2C(F)(F)F)C1)N1CCN(c2ncc(C(F)(F)F)cn2)CC1] |
| I-A-21 | (S)-4-(trifluoromethyl)-5-((4-(3-(4-(5- (trifluoromethyl)pyrimidin-2- yl)piperazine-1-carbonyl)azetidin-1- yl)butan-2-yl)amino)pyridazin-3(2H)- one | 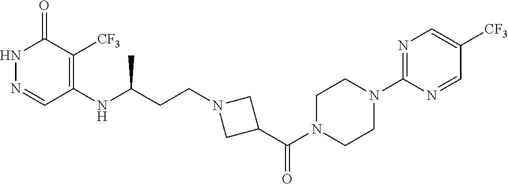 [SMILES: C[C@@H](CCN1CC(C(=O)N2CCN(c3ncc(C(F)(F)F)cn3)CC2)C1)Nc1cn[nH]c(=O)c1C(F)(F)F] |
| I-A-22 | (S)-4-chloro-5-((1-(3-(4-(5- (trifluoromethyl)pyrimidin-2- yl)piperazine-1-carbonyl)azetidin-1- yl)propan-2-yl)amino)pyridazin-3(2H)- one | 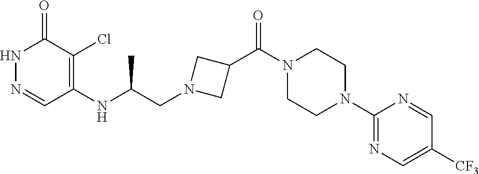 [SMILES: C[C@@H](CN1CC(C(=O)N2CCN(c3ncc(C(F)(F)F)cn3)CC2)C1)Nc1cn[nH]c(=O)c1Cl] |
| I-A-23 | (S)-4-bromo-5-((1-(3-(4-(5- (trifluoromethyl)pyrimidin-2- yl)piperazine-1-carbonyl)azetidin-1- yl)propan-2-yl)amino)pyridazin-3(2H)- one | 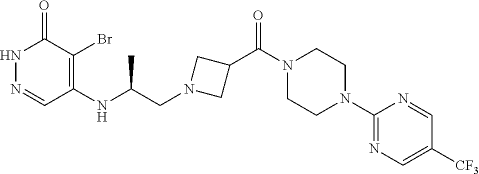 [SMILES: C[C@@H](CN1CC(C(=O)N2CCN(c3ncc(C(F)(F)F)cn3)CC2)C1)Nc1cn[nH]c(=O)c1Br] |
| I-A-24 | 4-(trifluoromethyl)-5-((1-((3-(4-(5- (trifluoromethyl)pyrimidin-2- yl)piperazine-1-carbonyl)azetidin-1- yl)methyl)cyclopropyl)amino)pyridazin- 3(2H)-one | 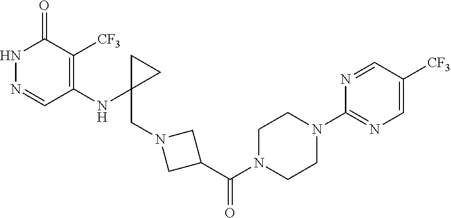 [SMILES: O=C(C1CN(CC2(Nc3cn[nH]c(=O)c3C(F)(F)F)CC2)C1)N1CCN(c2ncc(C(F)(F)F)cn2)CC1] |
| I-B-1 | (S)-5-((1-(3-(4-(2,2- difluorobenzo[d][1,3]dioxol-5- yl)piperazine-1-carbonyl)azetidin-1- yl)propan-2-yl)amino)-4- (trifluoromethyl)pyridazin-3(2H)-one | 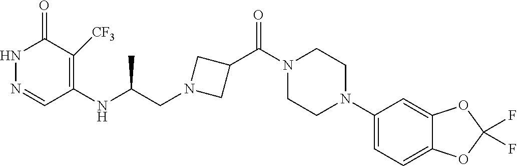 [SMILES: C[C@@H](CN1CC(C(=O)N2CCN(c3ccc4c(c3)OC(F)(F)O4)CC2)C1)Nc1cn[nH]c(=O)c1C(F)(F)F] |
| I-C-1 | (S)-4-(trifluoromethyl)-5-((1-(3-(4-(5- (trifluoromethyl)pyrimidin-2- yl)piperazine-1-carbonyl)-1H-pyrrol-1- yl)propan-2-yl)amino)pyridazin-3(2H)- one | 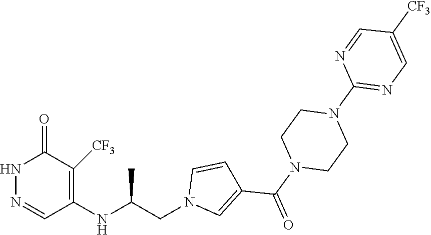 [SMILES: C[C@@H](Cn1ccc(C(=O)N2CCN(c3ncc(C(F)(F)F)cn3)CC2)c1)Nc1cn[nH]c(=O)c1C(F)(F)F] |
| I-D-1 | 4-(trifluoromethyl)-5-((1-(1-(1-(5- (trifluoromethyl)pyrimidin-2- yl)piperidine-4-carbonyl)azetidin-3- yl)propan-2-yl)amino)pyridazin-3(2H)- one | 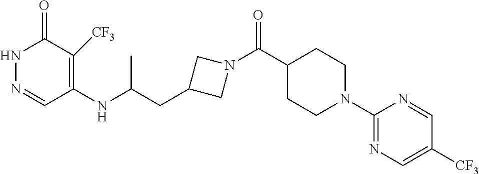 [SMILES: CC(CC1CN(C(=O)C2CCN(c3ncc(C(F)(F)F)cn3)CC2)C1)Nc1cn[nH]c(=O)c1C(F)(F)F] |
| I-E-1 | N-(4-((6-oxo-5-(trifluoromethyl)-1,6- dihydropyridazin-4-yl)amino)butyl)-4- (5-(trifluoromethyl)pyrimidin-2- yl)piperazine-1-carboxamide | 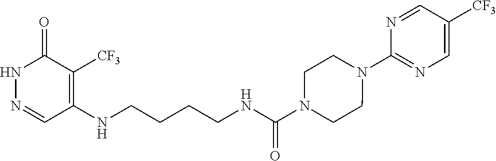 [SMILES: O=C(NCCCCNc1cn[nH]c(=O)c1C(F)(F)F)N1CCN(c2ncc(C(F)(F)F)cn2)CC1] |
| I-E-2 | (E)-N-(4-((6-oxo-5-(trifluoromethyl)- 1,6-dihydropyridazin-4-yl)amino)but-2- en-1-yl)-4-(5- (trifluoromethyl)pyrimidin-2- yl)piperazine-1-carboxamide | 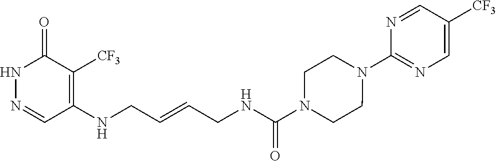 [SMILES: O=C(NC/C=C/CNc1cn[nH]c(=O)c1C(F)(F)F)N1CCN(c2ncc(C(F)(F)F)cn2)CC1] |
| I-E-3 | N-(3-((6-oxo-5-(trifluoromethyl)-1,6- dihydropyridazin-4-yl)amino)propyl)- 4-(2-(trifluoromethyl)pyrimidin-5- yl)piperazine-1-carboxamide | 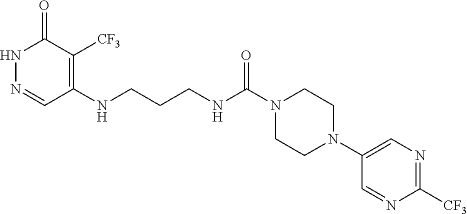 [SMILES: O=C(NCCCNc1cn[nH]c(=O)c1C(F)(F)F)N1CCN(c2cnc(C(F)(F)F)nc2)CC1] |
| I-E-4 | N-(2-((6-oxo-5-(trifluoromethyl)-1,6- dihydropyridazin-4-yl)amino)ethyl)-4- (2-(trifluoromethyl)pyrimidin-5- yl)piperazine-1-carboxamide | 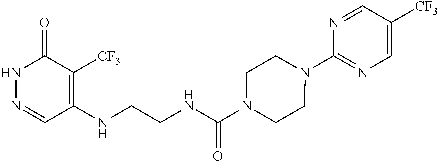 [SMILES: O=C(NCCNc1cn[nH]c(=O)c1C(F)(F)F)N1CCN(c2ncc(C(F)(F)F)cn2)CC1] |
| I-E-5 | N-(3-((6-oxo-5-(trifluoromethyl)-1,6- dihydropyridazin-4-yl)amino)propyl)- 4-(pyrimidin-5-yl)piperazine-1- carboxamide | 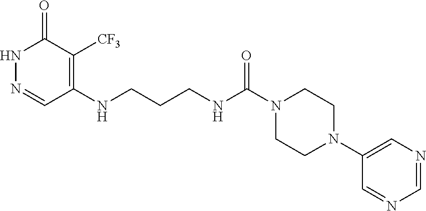 [SMILES: O=C(NCCCNc1cn[nH]c(=O)c1C(F)(F)F)N1CCN(c2cncnc2)CC1] |
| I-E-6 | N-(2-((6-oxo-5-(trifluoromethyl)-1,6- dihydropyridazin-4-yl)amino)ethyl)-4- (pyrimidin-5-yl)piperazine-1- carboxamide | 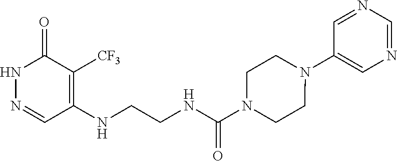 [SMILES: O=C(NCCNc1cn[nH]c(=O)c1C(F)(F)F)N1CCN(c2cncnc2)CC1] |
| I-E-7 | N-(3-(((6-oxo-5-(trifluoromethyl)-1,6- dihydropyridazin-4- yl)amino)methyl)phenyl)-4-(5- (trifluoromethyl)pyrimidin-2- yl)piperazine-1-carboxamide | 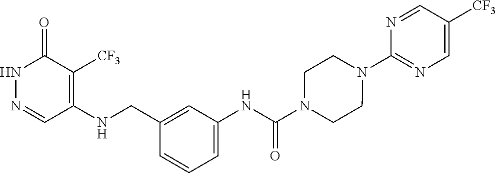 [SMILES: O=C(Nc1cccc(CNc2cn[nH]c(=O)c2C(F)(F)F)c1)N1CCN(c2ncc(C(F)(F)F)cn2)CC1] |

The pharmaceutically acceptable salt of the pyridazinone compound is a salt formed by the pyridazinone compound and an acid, and the acid is specifically hydrochloric acid, hydrobromic acid, sulfuric acid, phosphoric acid, carbonic acid, methanesulfonic acid, benzenesulfonic acid, p-toluenesulfonic acid, naphthalene sulfonic acid, citric acid, tartaric acid, lactic acid, pyruvic acid, acetic acid, maleic acid, succinic acid, fumaric acid, salicylic acid, phenylacetic acid, mandelic acid or ferulic acid.

As a second aspect of the present invention, a preparation method of the pyridazinone compound is any of the following methods:

(1) when R¹ is selected from trifluoromethyl, cyano or halogen, R² is hydrogen, R³ is selected from methyl or hydrogen, the carbon atom connected to R³ is the S configuration, or R² and R³ form a cyclopropyl, R⁴ is

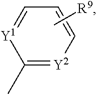

A¹ represents —NH—, A² is

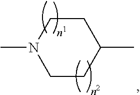

n¹ or n² is independently selected from 0 or 1, A³ is

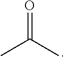

and A⁴ is

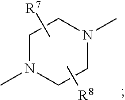

and a preparation method of a target compound I-A is as follows:

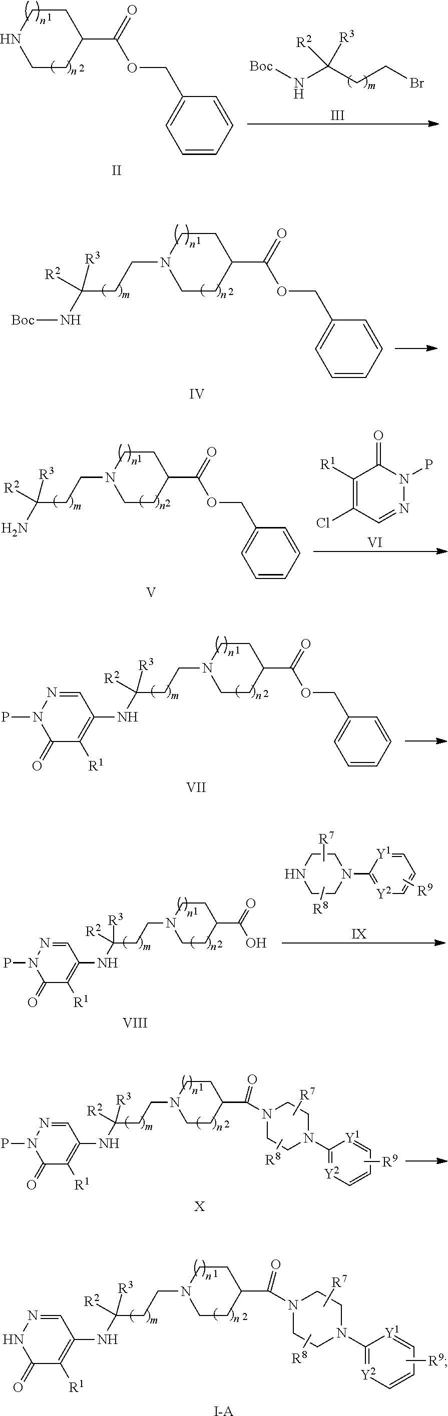

Compound IV is prepared from Compound II by dissolving Compounds II and III in a solvent, and an acid binding agent is added to perform a substitution reaction. The solvent is N,N-dimethylformamide, NN-dimethylacetamide, tetrahydrofuran, 1,4-dioxane, ethylene glycol dimethyl ether or acetonitrile, and preferably acetonitrile; and the acid binding agent is sodium carbonate, potassium carbonate, triethylamine or N,N-diisopropylethylamine (DIPEA), and preferably DIPEA.

Compound V is prepared from Compound IV by dissolving Compound IV in a solvent and adding an acid for reaction. The solvent is dichloromethane, tetrahydrofuran, and 1,4-dioxane, and preferably dichloromethane; and the acid is an ethyl acetate solution saturated with hydrogen chloride, a 1,4-dioxane solution saturated with hydrogen chloride or trifluoroacetic acid, preferably trifluoroacetic acid.

Compound VII is prepared from Compound V by dissolving Compounds V and VI in a solvent, and an acid binding agent is added to perform a substitution reaction. The solvent is N,N-dimethylformamide, NN-dimethylacetamide, tetrahydrofuran, 1,4-dioxane, ethylene glycol dimethyl ether or acetonitrile, and preferably 1,4-dioxane; and the acid binding agent is sodium carbonate, potassium carbonate, triethylamine or DIPEA, and preferably DIPEA.

Compound VIII is prepared from Compound VII by dissolving Compound VII in a solvent and adding a catalyst to carry out a catalytic hydrogenation reaction. The solvent is tetrahydrofuran, methanol, ethanol or a mixed solvent of any two of them, and preferably methanol; and the catalyst is palladium hydroxide or palladium carbon, and preferably palladium carbon.

Compound X is prepared from compound VIII by dissolving Compound VIII in a solvent, adding a condensation agent, and then adding an alkali and Compound IX to carry out a condensation reaction. The solvent is dichloromethane, tetrahydrofuran, N, N-dimethylformamide, 1,4-dioxane, ethylene glycol dimethyl ether or acetonitrile, and preferably N, N-dimethylformamide; the condensation agent is selected from N,N′-carbonyldiimidazole (CDI), 1-(3-dimethylaminopropyl)-3-ethylcarbodiimide hydrochloride (EDCI), N,N′-dicyclohexylcarbodiimide (DCC), N,N′-diisopropylcarbodiimide (DIC), 2-(7-azabenzotriazole)-N,N,N′N′-tetramethylurea hexafluorophosphate (HATU), Benzotriazole-N,N,N,N′-tetramethyluronium hexafluorophosphate (HBTU) or Benzotriazole-1-yl-oxytripyrrolidinophosphonium hexafluorophosphate (PyBop), and preferably EDCI; and the alkali is triethylamine, sodium bicarbonate, sodium carbonate, potassium carbonate or DIPEA, and preferably DIPEA.

Target compound I-A is prepared from Compound X by dissolving Compound X in a solvent and adding an acid for reaction. The solvent is tetrahydrofuran, acetonitrile or dichloromethane, and preferably dichloromethane; and the acid is hydrochloric acid or trifluoroacetic acid or trifluoromethanesulfonic acid, and preferably trifluoroacetic acid or trifluoromethanesulfonic acid.

(2) When R¹ is selected from trifluoromethyl, cyano or halogen, R² is hydrogen, R³ is selected from methyl or hydrogen, the carbon atom connected to R³ is the S configuration, or R² and R³ form a cyclopropyl, R⁴ is

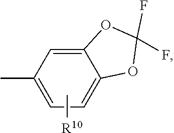

A¹ is —NH—, A² is

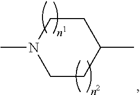

n¹ or n² is independently selected from 0 or 1, A³ is

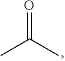

and A⁴ is

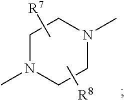

and a preparation method of a target compound I-B is as follows:

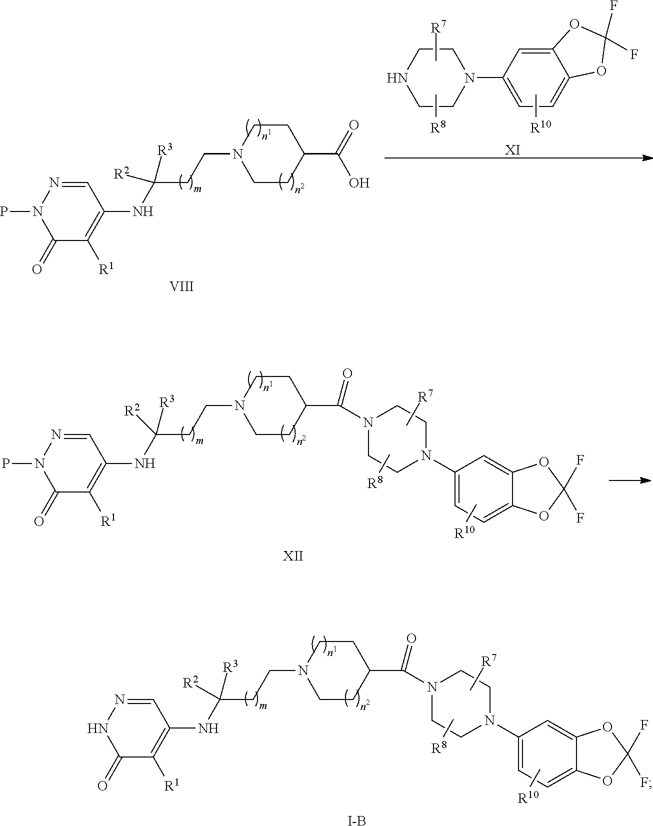

Compound XII is prepared from compound VIII by dissolving Compound VIII in a solvent, adding a condensation agent, and then adding an alkali and Compound XI to carry out a condensation reaction. The solvent is dichloromethane, tetrahydrofuran, N,N-dimethylformamide, 1,4-dioxane or acetonitrile, and preferably N,N-dimethylformamide; the condensation agent is selected from N,N′-carbonyldiimidazole (CDI), 1-(3-dimethylaminopropyl)-3-ethylcarbodiimide hydrochloride (EDCI), N,N′-dicyclohexylcarbodiimide (DCC), N,N′-diisopropylcarbodiimide (DIC), 2-(7-azabenzotriazole)-N,N,N′,N′-tetramethylurea hexafluorophosphate (HATU), Benzotriazole-N,N,N′,N′-tetramethyluronium hexafluorophosphate (HBTU) or Benzotriazole-1-yl-oxytripyrrolidinophosphonium hexafluorophosphate (PyBop), and preferably EDCI; and the alkali is triethylamine, sodium bicarbonate, sodium carbonate, potassium carbonate or DIPEA, and preferably DIPEA.

Target compound I-B is prepared from Compound XII by dissolving Compound XII in a solvent and adding an acid for reaction. The solvent is tetrahydrofuran, acetonitrile or dichloromethane, and preferably dichloromethane; and the acid is hydrochloric acid or trifluoroacetic acid or trifluoromethanesulfonic acid, and preferably trifluoroacetic acid or trifluoromethanesulfonic acid.

(3) when R¹ represents trifluoromethyl, cyano or halogen, R² is hydrogen, R³ is selected from methyl or hydrogen, the carbon atom connected to R³ is the S configuration, or R² and R³ form a cyclopropyl, R⁴ is

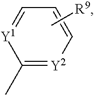

A¹ is —NH—, A² is

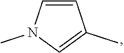

A³ is

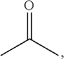

and A⁴ is

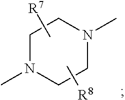

and a preparation method of a target compound I-C is as follows:

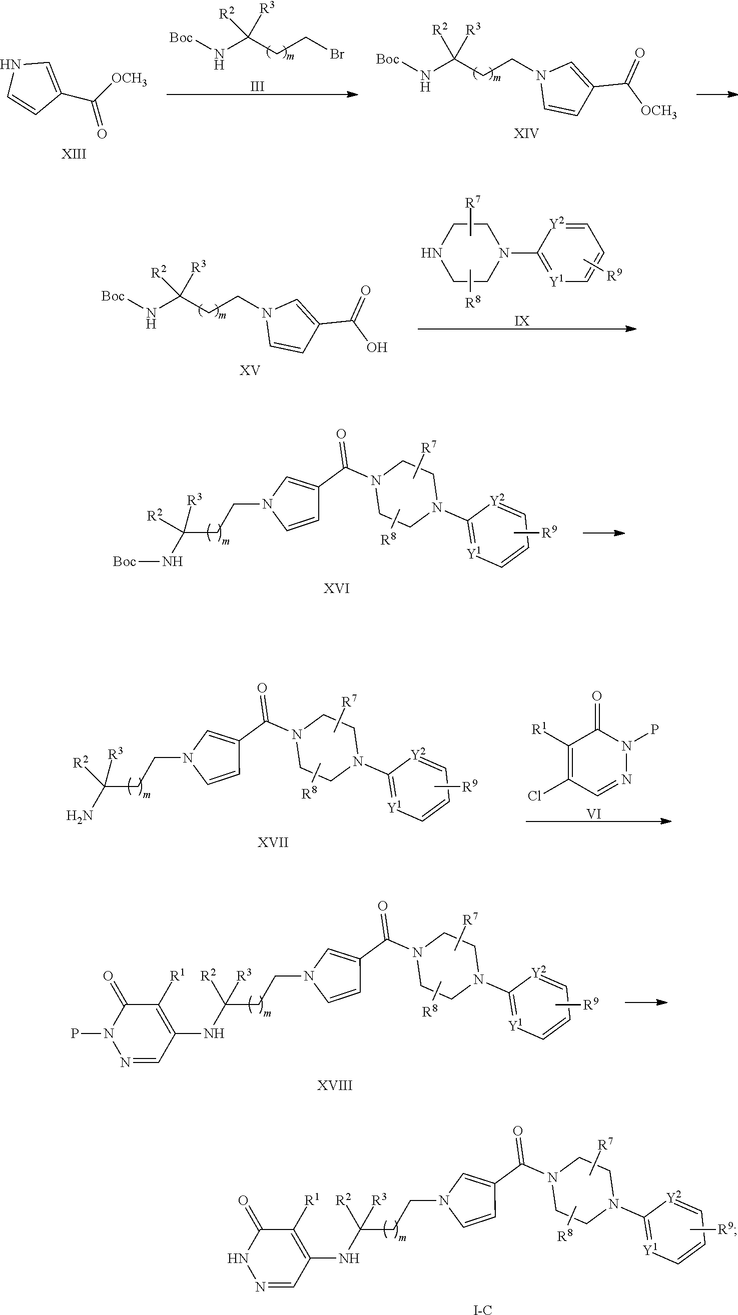

Compound XIV is prepared from Compound XIII by dissolving Compound XIII in a solvent, adding a base, and then adding Compound III for reaction. The solvent is N,N-dimethylformamide, NN-dimethylacetamide, tetrahydrofuran, 1,4-dioxane, ethylene glycol dimethyl ether or acetonitrile, and preferably N,N-dimethylformamide; and the alkali is sodium hydride, sodium carbonate, potassium carbonate, triethylamine or DIPEA, and preferably sodium hydride.

Compound XV is prepared from compound XIV by dissolving XIV in a solvent and adding an aqueous solution of an alkali for hydrolyzation. The solvent is tetrahydrofuran, methanol, acetonitrile or a mixed solvent of any two of them, and preferably a mixed solvent of tetrahydrofuran and methanol; and the alkali is sodium hydroxide, lithium hydroxide or potassium hydroxide, preferably sodium hydroxide.

Compound XVI is prepared from compound XV by dissolving Compound XV in a solvent, adding a condensation agent, and then adding an alkali and Compound IX to carry out a condensation reaction. The solvent is dichloromethane, tetrahydrofuran, N, N-dimethylformamide, 1,4-dioxane, ethylene glycol dimethyl ether or acetonitrile, and preferably N, N-dimethylformamide; the condensation agent is selected from N,N′-carbonyldiimidazole (CDI), 1-(3-dimethylaminopropyl)-3-ethylcarbodiimide hydrochloride (EDCI), N,N′-dicyclohexylcarbodiimide (DCC), N,N′-diisopropylcarbodiimide (DIC), 2-(7-azabenzotriazole)-N,N,N′,N-tetramethylurea hexafluorophosphate (HATU), Benzotriazole-N,N,N,N′-tetramethyluronium hexafluorophosphate (HBTU) or Benzotriazole-1-yl-oxytripyrrolidinophosphonium hexafluorophosphate (PyBop), and preferably EDCI; and the alkali is triethylamine, sodium carbonate, potassium carbonate or DIPEA, and preferably DIPEA.

Compound XVII is prepared from Compound XVI by dissolving Compound XVI in a solvent and adding an acid for reaction. The solvent is dichloromethane, tetrahydrofuran or 1,4-dioxane, and preferably dichloromethane; and the acid is hydrochloric acid, trifluoroacetic acid or sulfuric acid, and preferably trifluoroacetic acid.

Compound XVIII is prepared from Compound XVII by dissolving Compound XVII and Compound VI in a solvent and adding an alkali to carry out a substitution reaction. The solvent is N,N-dimethylformamide, NN-dimethylacetamide, tetrahydrofuran, 1,4-dioxane, ethylene glycol dimethyl ether or acetonitrile, and preferably 1,4-dioxane; and the alkali is sodium carbonate, potassium carbonate, triethylamine or DIPEA, and preferably DIPEA.

Compound I-C is prepared from Compound XVIII by dissolving Compound XVIII in a solvent and adding an acid for reaction. The solvent is tetrahydrofuran, acetonitrile or dichloromethane, and preferably dichloromethane; and the acid is hydrochloric acid or trifluoroacetic acid or trifluoromethanesulfonic acid, and preferably trifluoroacetic acid or trifluoromethanesulfonic acid.

(4) when m=0, R¹ is selected from trifluoromethyl, cyano or halogen, R² is hydrogen, R³ is methyl, R⁴ is

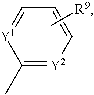

A¹ is —NH—, A² is

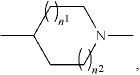

n¹ or n² is independently selected from 0 or 1, A³ is

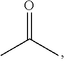

and A⁴ is

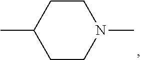

and a preparation method of a target compound I-D is as follows:

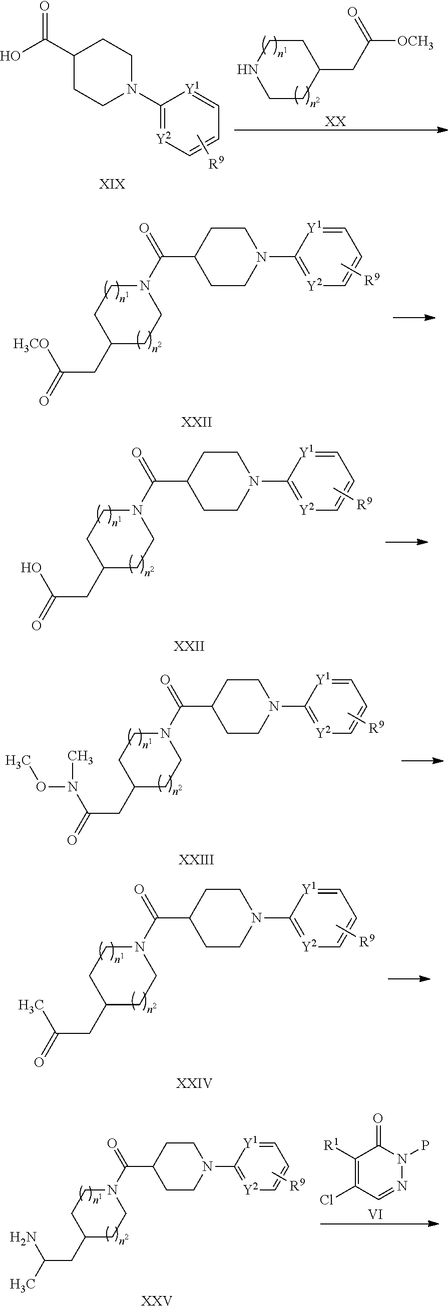

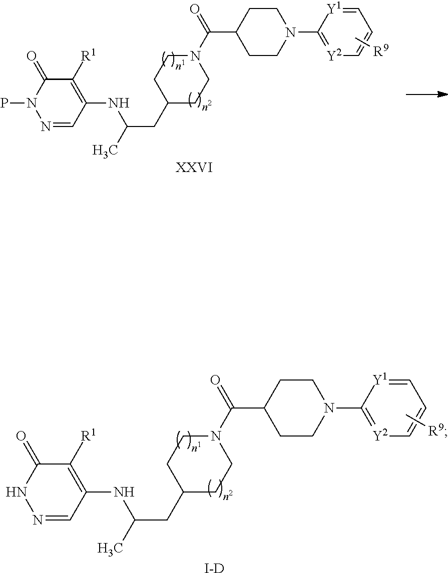

Compound XXI is prepared from Compound XIX by dissolving Compound XIX in a solvent, adding a condensation agent, and then adding an alkali and Compound XX to carry out a condensation reaction. The solvent is dichloromethane, tetrahydrofuran, N, N-dimethylformamide, 1,4-dioxane, ethylene glycol dimethyl ether or acetonitrile, and preferably N, N-dimethylformamide; the condensation agent is selected from N,N′-carbonyldiimidazole (CDI), 1-(3-dimethylaminopropyl)-3-ethylcarbodiimide hydrochloride (EDCI), N,N′-dicyclohexylcarbodiimide (DCC), N,N′-diisopropylcarbodiimide (DIC), 2-(7-azabenzotriazole)-N,N,N′,N′-tetramethylurea hexafluorophosphate (HATU), Benzotriazole-N,N,N,N′-tetramethyluronium hexafluorophosphate (HBTU) or Benzotriazole-1-yl-oxytripyrrolidinophosphonium hexafluorophosphate (PyBop), and preferably EDCI; and the alkali is triethylamine, sodium carbonate, potassium carbonate or DIPEA, and preferably DIPEA.

Compound XXII is prepared from Compound XXI by dissolving Compound XXI in a solvent and adding an aqueous solution of an alkali for hydrolyzation. The solvent is tetrahydrofuran, methanol, acetonitrile or a mixed solvent of any two of them, and preferably a mixed solvent of tetrahydrofuran and methanol; and the alkali is sodium hydroxide, lithium hydroxide or potassium hydroxide, preferably sodium hydroxide.

Compound XXIII is prepared from Compound XXII by dissolving Compound XXII in a solvent, adding a condensation agent, and then adding N, O-dimethylhydroxylamine hydrochloride carry out a condensation reaction. The solvent is dichloromethane, tetrahydrofuran, N, N-dimethylformamide, 1,4-dioxane, ethylene glycol dimethyl ether or acetonitrile, and preferably N, N-dimethylformamide; the condensation agent is selected from N,N′-carbonyldiimidazole (CDI), 1-(3-dimethylaminopropyl)-3-ethylcarbodiimide hydrochloride (EDCI), N,N′-dicyclohexylcarbodiimide (DCC), N,N′-diisopropylcarbodiimide (DIC), 2-(7-azabenzotriazole)-N,N,N′,N′-tetramethylurea hexafluorophosphate (HATU), Benzotriazole-N,N,N,N′-tetramethyluronium hexafluorophosphate (HBTU) or Benzotriazole-1-yl-oxytripyrrolidinophosphonium hexafluorophosphate (PyBop), and preferably CDI.

Compound XXIV is prepared from Compound XXIII by dissolving Compound XXIII in a solvent and adding methylmagnesium bromide at a low temperature for reaction. The solvent is anhydrous tetrahydrofuran, anhydrous ether, anhydrous dichloromethane or anhydrous dioxane, and preferably anhydrous tetrahydrofuran.

Compound XXV is prepared from Compound XXIV by dissolving Compound XXIV in a solvent and adding an ammonia source and a catalyst for reaction. The solvent is methanol, ethanol, tetrahydrofuran or a mixed solvent of any two of them, and preferably methanol; the ammonia source is ammonium formate or ammonium acetate, and preferably ammonium formate; and the catalyst is preferably dichloro(pentamethylcyclopentadienyl)rhodium (III) dimer.

Compound XXVI is prepared from Compound XXV by dissolving Compound XXV and Compound VI in a solvent and adding an alkali to carry out a substitution reaction. The solvent is N,N-dimethylformamide, NN-dimethylacetamide, tetrahydrofuran, 1,4-dioxane, ethylene glycol dimethyl ether or acetonitrile, and preferably 1,4-dioxane; and the alkali is sodium carbonate, potassium carbonate, triethylamine or DIPEA, and preferably DIPEA.

Target compound I-D is prepared from Compound XXVI by dissolving Compound XXVI in a solvent and adding an acid for reaction. The solvent is tetrahydrofuran, acetonitrile, dichloromethane or a mixed solvent of any two of them, and preferably dichloromethane; and the acid is hydrochloric acid or trifluoroacetic acid or trifluoromethanesulfonic acid, and preferably trifluoroacetic acid or trifluoromethanesulfonic acid.

(5) when R¹ is selected from trifluoromethyl, cyano or halogen, R² and R³ are hydrogen, R⁴ is

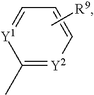

A¹ represents —NH—, A² is —NH— or —N(CH₃)—, A³ is

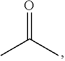

and A⁴ is

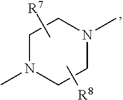

and a preparation method of a target compound I-E is as follows:

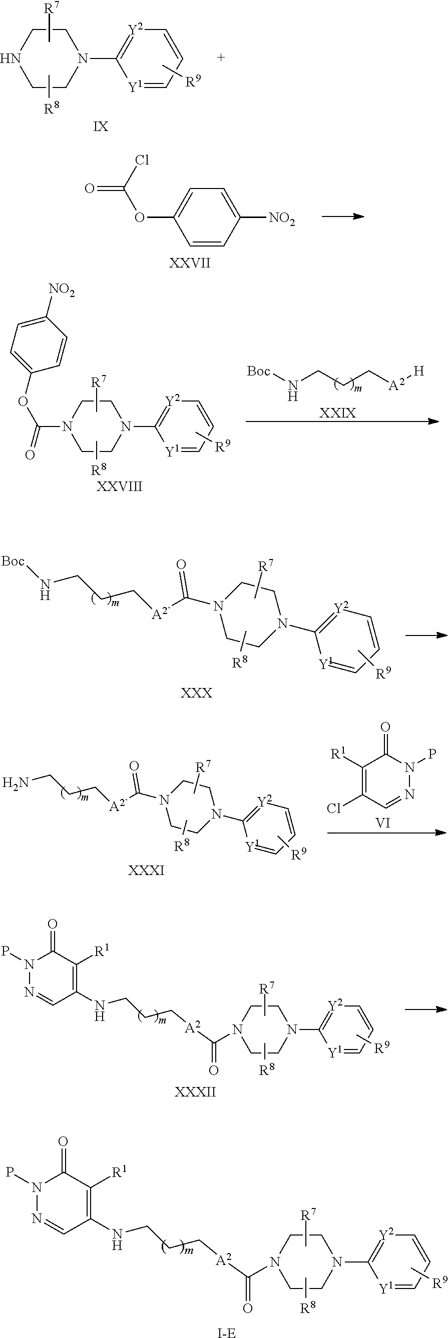

Compound XXVIII is prepared from Compound IX by dissolving Compound IX in a solvent, adding an alkali, and then adding Compound XXVII for reaction. The solvent is N,N-dimethylformamide, N,N-dimethylacetamide, tetrahydrofuran, 1,4-dioxane, dichloromethane or acetonitrile, and preferably dichloromethane; and the alkali is sodium carbonate, potassium carbonate, triethylamine or DIPEA, and preferably triethylamine.

Compound XXX can be prepared from Compound XVIII by dissolving Compound XVIII in a solvent, adding Compound XXIX, and then adding an alkali for action. The solvent is tetrahydrofuran, N,N-dimethylformamide, N,N-dimethylacetamide or acetonitrile, and preferably N,N-dimethylformamide; and the alkali is sodium hydroxide, potassium carbonate or sodium carbonate, and preferably potassium carbonate.

Compound XXXI is prepared from Compound XXX by dissolving Compound XXX in a solvent and adding an acid for reaction. The solvent is dichloromethane, tetrahydrofuran or 1,4-dioxane, and preferably dichloromethane; and the acid is hydrochloric acid, trifluoroacetic acid or sulfuric acid, and preferably trifluoroacetic acid.

Compound XXXII is prepared from Compound XXXI by dissolving Compounds XXXI and VI in a solvent and adding an alkali to carry out a substitution reaction. The solvent is N,N-dimethylformamide, NN-dimethylacetamide, tetrahydrofuran, 1,4-dioxane, ethylene glycol dimethyl ether or acetonitrile, and preferably 1,4-dioxane; and the alkali is sodium carbonate, potassium carbonate, triethylamine or DIPEA, and preferably DIPEA.

Target compound I-E is prepared from Compound XXXII by dissolving Compound XXXII in a solvent and adding an acid for reaction. The solvent is dichloromethane, tetrahydrofuran or 1,4-dioxane, and preferably dichloromethane; and the acid is hydrochloric acid, trifluoroacetic acid or sulfuric acid, and preferably trifluoroacetic acid.

Specifically, m, Y¹, Y², R⁷, R⁸, R⁹ and R¹⁰ are defined as stated above; Boc is tert-butoxycarbonyl; P is (trimethylsilyl) ethoxymethyl (SEM) or p-methoxybenzyl (PMB); and
the corresponding acid is prepared into a salt with Compound (I) prepared by the above method to obtain the pharmaceutically acceptable salt of the pyridazinone compound.

As a third aspect of the present invention, the pharmaceutical composition includes any of the pyridazinone compounds and the pharmaceutically acceptable salt. Pharmaceutically acceptable carriers can be added to prepare common pharmaceutical formulations, such as tablets, capsules, syrups, suspensions, or injections. The formulations can be added with flavors, sweeteners, liquid/solid fillers, diluents, and other common pharmaceutical excipients.

As a fourth aspect of the present invention, the pyridazinone compound and the pharmaceutical composition thereof can be prepared as PARP7 inhibitor drugs, specifically drugs for treating tumors, and more specifically drugs for treating lung squamous carcinoma, colon cancer, breast cancer and other cancers.

Beneficial effects: compared with the prior art, the present invention has the following advantages:

1. The pyridazinone compound has excellent in vivo pharmacokinetic properties, significantly improved half-life, in vivo exposure, and bioavailability, exhibiting remarkable drug-forming advantages. In addition, the pyridazinone compound exhibits excellent in vivo pharmacodynamic properties, and can achieve significant tumor growth inhibition activity at a lower dose. Further, the pyridazinone compound can promote the release of immune factors, and can significant therapeutic effects without the need for drug combination.

2. The pyridazinone compound can effectively inhibit PARP7 enzyme activity, with the optimal enzyme inhibition IC₅₀ value being less than 0.1 μM, reaching nanomolar concentration level.

3. The pyrazinone compound and the pharmaceutical composition thereof have wide applications and can be prepared as anti-tumor drugs, exhibiting more excellent pharmacokinetic and pharmacokinetic properties.

The preparation method for the pyrazinone compound is simple and easy.

### BRIEF DESCRIPTION OF THE DRAWINGS

FIG. 1 illustrates shows an effect of compounds on interferon release.

FIG. 2 illustrates in vivo anti-tumor effects of compounds.

### DETAILED DESCRIPTIONS OF THE EMBODIMENTS

The technical solution of the present invention will be further described below with reference to the embodiments.

### Example 1: Synthesis of (S)-4-(trifluoromethyl)-5-((1-(3-(4-(5-(trifluoromethyl)pyrimidin-2-yl)piperazine-1-carbonyl)azetidin-1-yl)propan-2-yl)amino)pyridazin-3(2H)-one (I-A-1)

### Synthesis of benzyl (S)-1-(2-((tert-butoxycarbonyl)amino)propyl)azetidine-3-carboxylate (IV-1)

Azetidine-3-carboxylate (II-1) (16.0 g, 83.7 mmol) was dissolved in 70 mL of acetonitrile, tert-butyl (S)-(1-bromopropan-2-yl)carbamate (III-1) (21.9 g, 92.0 mmol) and DIPEA (32.4 g, 251.1 mmol) were added to obtain a mixture, a temperature of the mixture was raised to 80° C. for reaction for 2 h. Thin layer chromatography (V petroleum ether:V ethyl acetate=1:1) was adopted to monitor the reaction, 200 mL of water was added, ethyl acetate (100 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=3:1) to obtain 25.4 g of colorless oil (IV-1), with a yield of 87.0%. ESI-MS [M+H]⁺ 349.2; ¹H NMR (300 MHz, DMSO-d₆) δ 7.40-7.30 (m, 5H), 5.14 (s, 2H), 3.65-3.50 (m, 3H), 3.40-3.30 (m, 3H), 2.45 (dd, J₁=6 Hz, J₂=1.8 Hz, 2H), 1.44 (s, 9H), 1.08 (d, J=6.6 Hz, 3H).

### Synthesis of benzyl (S)-1-(2-aminopropyl)azetidine-3-carboxylate (V-1)

Compound IV-1 (6.9 g, 20.0 mmol) was dissolved in 30 mL of dichloromethane, 20 mL of trifluoroacetic acid was added to obtain a mixture, the mixture was stirred at a room temperature for 0.5 h, and thin layer chromatography (V dichloromethane:V methanol=15:1) was adopted to monitor the reaction, a solvent therein was evaporated and removed under reduced pressure, a saturated aqueous sodium bicarbonate solution was added to adjust a pH to 7-8. Dichloromethane (50 mL×7) was used for extraction, organic phases were combined, and then washed with saturated brine, dried over anhydrous sodium sulfate and filtered to obtain a filtrate, and the filtrate was concentrated to obtain 4.40 g of light yellow oil (V-1), with a yield of 88.7%. ESI-MS [M+H]⁺ 249.2.

### Synthesis of benzyl (S)-1-(2-((6-oxo-5-(trifluoromethyl)-1-((2-(trimethylsilyl)ethoxy)methyl)-1,6-dihydropyridazin-4-yl)amino)propyl)azetidine-3-carboxylate (VII-1)

Compound V-1 (2.2 g, 9.0 mmol) was dissolved in 10 mL of 1,4-dioxane, 5-chloro-4-(trifluoromethyl)-2-((2-(trimethylsilyl)ethoxy)methyl)pyridazin-3(2H)-one (VI-1) (3.3 g, 10.0 mmol) and DIPEA (3.5 g, 27.0 mmol) were added to obtain a mixture, a temperature of the mixture was raised to 90° C. for reaction for 2 h; and thin layer chromatography (V petroleum ether:V ethyl acetate=1:1) was adopted to monitor the reaction, 40 mL of water was added, ethyl acetate (20 mL×3) was used for extraction, organic phases were combined, and then washed with saturated brine in sequence, dried over anhydrous sodium sulfate, filtered and concentrated. A crude product was purified by silica gel column chromatography (V petroleum ether:V ethyl acetate=3:1) to obtain 4.1 g of colorless oil (VII-1), with a yield of 83.7%. ESI-MS [M+H]⁺ 541.2; ¹H NMR (300 MHz, DMSO-d₆) δ 7.61 (s, 1H), 7.38-7.32 (m, 5H), 6.15-6.05 (m, 1H), 5.37 (dd, J₁=14.1 Hz, J₂=9.9 Hz, 2H), 5.14 (s, 2H), 3.74-3.55 (m, 5H), 3.45-3.35 (m, 3H), 2.72-2.54 (m, 2H), 1.23 (d, J=6.6 Hz, 3H), 0.99-0.91 (m, 2H), −0.01 (s, 9H).

### Synthesis of (S)-1-(2-((6-oxo-5-(trifluoromethyl)-1-((2-(trimethylsilyl)ethoxy)methyl)-1,6-dihydropyridazin-4-yl)amino)propyl)azetidine-3-carboxylic acid (VIII-1)

Compound VII-1 (4.0 g, 7.4 mmol) was dissolved in 20 mL of anhydrous methanol, 500 mg of palladium carbon was added, after a reaction system was replaced with hydrogen, and the reaction was performed at the room temperature for 30 min; and thin layer chromatography (V dichloromethane:V methanol=15:1) was adopted to monitor the reaction, the mixture was subjected to suction filtration to obtain a filter cake, and the filter cake was washed with 10 mL of methanol, and dried to obtain 3.2 g of white solid (VIII-1), with a yield of 96.1%. ESI-MS [M+H]⁺ 451.2; ¹H NMR (300 MHz, DMSO-d₆) δ 11.4 (s, 1H), 7.62 (s, 1H), 6.45-6.35 (m, 1H), 5.37 (dd, J₁=14.1 Hz, J₂=9.9 Hz, 2H), 3.74-3.55 (m, 5H), 3.45-3.35 (m, 3H), 2.72-2.54 (m, 2H), 1.23 (d, J=6.6 Hz, 3H), 0.99-0.91 (m, 2H), −0.01 (s, 9H).

Synthesis of (S)-4-(trifluoromethyl)-5-((1-(3-(4-(5-(trifluoromethyl)pyrimidin-2-yl)piperazine-1-carbonyl)azetidin-1-yl)propan-2-yl)amino)-2-((2-(trimethylsilyl)ethoxy)methyl)pyridazin-3(2H)-one (X-1)

Compound VIII-1 (480.0 mg, 1.1 mmol) was dissolved in 5 mL of dichloromethane, 1-Hydroxybenzotriazole (HOBt, 180.0 mg, 1.3 mmol) and EDCI (210.0 mg, 1.3 mmol) were added respectively to obtain a mixture, the mixture was reacted at room temperature for 0.5 h, and 2-(piperazin-1-yl)-5-(trifluoromethyl)pyrimidine (IX-1) (280.0 mg, 1.2 mmol) and DIPEA (430.0 mg, 3.3 mmol) were added in sequence to allow the mixture to continuously react at the room temperature for 1 h, and thin layer chromatography (V dichloromethane:V methanol=15:1) was adopted to monitor the reaction. 10 mL of water was added, dichloromethane (5 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=1:2) to obtain 560.0 mg of a white solid (X-1), with a yield of 76.6%. ESI-MS [M+H]⁺ 665.2; ¹H NMR (300 MHz, DMSO-d₆) δ 8.73 (s, 2H), 8.00 (s, 1H), 6.65-6.55 (m, 1H), 5.21 (s, 2H), 4.20-4.00 (m, 1H), 3.83-3.74 (m, 4H), 3.66-3.45 (m, 8H), 3.28-3.13 (m, 5H), 1.24 (t, J=7.2 Hz, 1H), 1.18 (d, J=6.3 Hz, 3H), 0.84 (t, J=8.1 Hz, 2H), −0.04 (s, 9H).

### Synthesis of (S)-4-(trifluoromethyl)-5-((1-(3-(4-(5-(trifluoromethyl)pyrimidin-2-yl)piperazine-1-carbonyl)azetidin-1-yl)propan-2-yl)amino)pyridazin-3(2H)-one (I-A-1)

Compound X-1 (531.2 mg, 0.8 mmol) was dissolved in 3 mL of dichloromethane, and 3 mL trifluoroacetic acid was added and reacted at the room temperature for 3 h. Thin layer chromatography (V dichloromethane:V methanol=15:1) was adopted to monitor the reaction, a solvent therein was evaporated and removed under reduced pressure, saturated aqueous sodium bicarbonate solution was added to adjust a pH value thereof to 7-8, 10 mL of water was added, dichloromethane (10 mL×3) was used for extraction, organic phases were combined, and then washed with saturated brine, dried over anhydrous sodium sulfate, filtered and concentrated. A crude product was purified by silica gel column chromatography (V dichloromethane:V methanol=40:1) to obtain 332.4 mg of a white solid (I-A-1), with a yield of 77.8%. ESI-MS [M+H]⁺ 535.2; ¹H NMR (300 MHz, DMSO-d₆) δ 12.50 (s, 1H), 8.73 (s, 2H), 7.90 (s, 1H), 6.49-6.35 (m, 1H), 4.10-3.90 (m, 1H), 3.88-3.72 (m, 4H), 3.62-3.42 (m, 11H), 1.16 (d, J=6.3 Hz, 3H).

With reference to the preparation method described in Example 1, the following compounds were prepared:

| Compound | ¹H-NMR | MS |
|---|---|---|
| I-A-2 | ¹H NMR (400 MHz, DMSO-d₆) δ 12.47 (s, 1H), 8.81 (s, 2H), | ESI-MS: [M + H]⁺ |
| | 7.89 (d, J = 8.4 Hz, 3H), 7.78 (d, J = 8.4 Hz, 4H), 6.45-6.35 | 611.2 |
| | (m, 1H), 4.00-3.90 (m, 1H), 3.82-3.74 (m, 4H), 3.62-3.52 (m, | |
| | 5H), 3.32-3.22 (m, 4H), 2.65-2.51 (m, 2H), 1.13 (d, J = 6.3 Hz, | |
| | 3H). | |
| I-A-3 | ¹H NMR (400 MHz, DMSO-d₆) δ 12.46 (s, 1H), 8.71 (s, 2H), | ESI-MS: [M + H]⁺ |
| | 7.81 (s, 1H), 6.43-6.35 (m, 1H), 4.00-3.90 (m, 1H), 3.80-3.71 | 492.2 |
| | (m, 4H), 3.60-3.50 (m, 5H), 3.32-3.12 (m, 4H), 2.65-2.51 (m, | |
| | 2H), 1.12 (d, J = 6.3 Hz, 3H). | |
| I-A-4 | ¹H NMR (400 MHz, DMSO-d₆) δ 12.46 (s, 1H), 8.73 (s, 2H), | ESI-MS: [M + H]⁺ |
| | 7.77 (s, 1H), 6.44-6.35 (m, 1H), 4.00-3.90 (m, 1H), 3.92-3.72 | 544.2 |
| | (m, 4H), 3.62-3.45 (m, 5H), 3.44-3.36 (m, 4H), 3.22 (s, 3H), | |
| | 2.70-2.55 (m, 2H), 1.13 (d, J = 6.3 Hz, 3H). | |
| I-A-5 | ¹H NMR (400 MHz, DMSO-d₆) δ 12.47 (s, 1H), 8.44 (s, 2H), | ESI-MS: [M + H]⁺ |
| | 7.89 (s, 1H), 6.46-6.35 (m, 1H), 4.00-3.90 (m, 1H), 3.72-3.64 | 501.2 |
| | (m, 4H), 3.56-3.48 (m, 5H), 3.34-3.30 (m, 2H), 3.28-3.18 (m, | |
| | 2H), 2.70-2.55 (m, 2H), 1.12 (d, J = 6.3 Hz, 3H). | |
| I-A-6 | ¹H NMR (400 MHz, DMSO-d₆) δ 12.47 (s, 1H), 8.24 (s, 2H), | ESI-MS: [M + H]⁺ |
| | 7.90 (s, 1H), 6.45-6.35 (m, 1H), 4.00-3.90 (m, 1H), 3.72-3.64 | 481.2 |
| | (m, 4H), 3.56-3.48 (m, 4H), 3.34-3.28 (m, 4H), 3.24-3.18 (m, | |
| | 1H), 2.70-2.55 (m, 2H), 2.08 (s, 3H), 1.12 (d, J = 6.3 Hz, 3H). | |
| I-A-7 | ¹H NMR (400 MHz, DMSO-d₆) δ 12.47 (s, 1H), 8.55 (s, 2H), | ESI-MS: [M + H]⁺ |
| | 7.96 (s, 1H), 6.45-6.35 (m, 1H), 4.00-3.90 (m, 1H), 3.72-3.62 | 545.1 |
| | (m, 4H), 3.56-3.46 (m, 5H), 3.43-3.36 (m, 3H), 3.26-3.16 (m, | |
| | 1H), 2.65-2.54 (m, 2H), 1.12 (d, J = 6.3 Hz, 3H). | |
| I-A-8 | ¹H NMR (400 MHz, DMSO-d₆) δ 12.45 (s, 1H), 8.46 (s, 2H), | ESI-MS: [M + H]⁺ |
| | 7.89 (s, 1H), 6.43-6.35 (m, 1H), 3.95-3.85 (m, 1H), 3.70-3.60 | 485.2 |
| | (m, 4H), 3.57-3.45 (m, 5H), 3.32-3.27 (m, 3H), 2.65-2.54 (m, | |
| | 2H), 1.12 (d, J = 6.3 Hz, 3H). | |
| I-A-9 | ¹H NMR (400 MHz, DMSO-d₆) δ 12.47 (s, 1H), 8.22 (s, 2H), | ESI-MS: [M + H]⁺ |
| | 7.89 (s, 1H), 6.44-6.35 (m, 1H), 3.95-3.85 (m, 1H), 3.77 (s, | 497.2 |
| | 3H), 3.65-3.45 (m, 10H), 3.32-3.27 (m, 3H), 2.65-2.54 (m, | |
| | 2H), 1.12 (d, J = 6.3 Hz, 3H). | |
| I-A-10 | ¹H NMR (400 MHz, DMSO-d₆) δ 12.46 (s, 1H), 8.46 (s, 1H), | ESI-MS: [M + H]⁺ |
| | 7.89 (s, 1H), 7.81 (dd, J₁ = 9.2 Hz, J₂= 2.8 Hz, 1H), 6.96 (d, J = | 534.2 |
| | 9.2 Hz, 1H), 6.45-6.35 (m, 1H), 4.00-3.90 (m, 1H), 3.69-3.45 | |
| | (m, 10H), 3.32-3.22 (m, 3H), 2.65-2.54 (m, 2H), 1.12 (d, J = | |
| | 6.3 Hz, 3H). | |
| I-A-11 | ¹H NMR (400 MHz, DMSO-d₆) δ 12.46 (s, 1H), 7.89 (s, 1H), | ESI-MS: [M + H]⁺ |
| | 7.51 (d, J = 8.8 Hz, 2H), 7.06 (d, J = 8.8 Hz, 2H), 6.46-6.35 | 533.2 |
| | (m, 1H), 4.00-3.90 (m, 1H), 3.64-3.45 (m, 5H), 3.43-3.35 (m, | |
| | 3H), 3.30-3.18 (m, 5H), 2.65-2.54 (m, 2H), 1.12 (d, J = 6.3 Hz, | |
| | 3H). | |
| I-A-12 | ¹H NMR (400 MHz, DMSO-d₆) δ 12.49 (s, 1H), 8.10 (d, J = | ESI-MS: [M + H]⁺ |
| | 7.6 Hz, 1H), 7.90 (s, 1H), 7.71 (t, J = 7.6 Hz, 2H), 7.51 (t, J = | 555.2 |
| | 7.6 Hz, 1H), 7.39 (t, J = 7.6 Hz, 1H), 7.29 (t, J = 7.6 Hz, 1H), | |
| | 6.99 (d, J = 7.9 Hz, 1H), 6.45-6.35 (m, 1H), 4.00-3.90 (m, 1H), | |
| | 3.75-3.65 (m, 2H), 3.62-3.45 (m, 6H), 3.33-3.15 (m, 6H), 2.65- | |
| | 2.54 (m, 2H), 1.13 (d, J = 6.3 Hz, 3H). | |
| I-A-13 | ¹H NMR (400 MHz, DMSO-d₆) δ 12.46 (s, 1H), 8.70 (d, J = | ESI-MS: [M + H]⁺ |
| | 4.8 Hz, 1H), 7.89 (s, 1H), 7.05 (d, J = 4.8 Hz, 1H), 6.44-6.35 | 535.2 |
| | (m, 1H), 4.00-3.90 (m, 1H), 3.75-3.65 (m, 4H), 3.62-3.45 (m, | |
| | 5H), 3.44-3.39 (m, 4H), 3.26-3.20 (m, 2H), 2.65-2.54 (m, 2H), | |
| | 1.12 (d, J = 6.3 Hz, 3H). | |
| I-A-14 | ¹H NMR (400 MHz, DMSO-d₆) δ 12.48 (s, 1H), 8.37 (d, J = | ESI-MS: [M + H]⁺ |
| | 4.8 Hz, 2H), 7.90 (s, 1H), 6.65 (t, J = 4.8 Hz, 1H), 6.46-6.35 | 467.2 |
| | (m, 1H), 4.00-3.90 (m, 1H), 3.75-3.65 (m, 4H), 3.55-3.45 (m, | |
| | 5H), 3.35-3.27 (m, 3H), 3.26-3.18 (m, 3H), 2.65-2.54 (m, 2H), | |
| | 1.12 (d, J = 6.3 Hz, 3H). | |
| I-A-15 | ¹H NMR (300 MHz, DMSO-d₆) δ 12.48 (s, 1H), 8.69 (s, 1H), | ESI-MS: [M + H]⁺ |
| | 8.65-8.55 (m, 1H), 8.50 (s, 1H), 7.89 (s, 1H), 6.48-6.36 (m, | 521.2 |
| | 1H), 4.71-4.54 (m, 1H), 4.36 (t, J = 8.1 Hz, 1H), 4.24 (t, J = | |
| | 8.1 Hz, 1H), 3.98-3.88 (m, 2H), 3.84-3.76 (m, 1H), 3.26-3.08 | |
| | (m, 4H), 1.30-1.20 (m, 3H), 1.12 (d, J = 6.3 Hz, 3H). | |
| I-A-16 | ¹H NMR (300 MHz, DMSO-d₆)) δ 12.48 (s, 1H), 8.69 (s, 1H), | ESI-MS: [M + H]+ |
| | 8.50 (s, 1H), 7.89 (s, 1H), 6.48-6.36 (m, 1H), 4.71-4.54 (m, | 535.2 |
| | 1H), 4.36 (t, J = 8.1 Hz, 1H), 4.24 (t, J = 8.1 Hz, 1H), 3.98- | |
| | 3.88 (m, 2H), 3.84-3.76 (m, 1H), 3.32 (s, 3H), 3.26-3.08 (m, | |
| | 4H), 1.30-1.20 (m, 3H), 1.12 (d, J = 6.3 Hz, 3H). | |
| I-A-17 | ¹H NMR (300 MHz, DMSO-d₆) δ 12.50 (s, 1H), 8.73 (s, 2H), | ESI-MS: [M + H]⁺ |
| | 7.90 (s, 1H), 6.49-6.35 (m, 1H), 4.10-3.90 (m, 1H), 3.92-3.72 | 563.2 |
| | (m, 4H), 3.62-3.42 (m, 9H), 1.16 (d, J = 6.3 Hz, 3H), 1.00 (d, | |
| | J = 6.4 Hz, 6H). | |

### Example 2: Synthesis of 4-(trifluoromethyl)-5-(((2S)-1-(3-(4-(5-(trifluoromethyl)pyrimidin-2-yl)piperazine-1-carbonyl)pyrrolidin-1-yl)propan-2-yl)amino)pyridazin-3(2H)-one (I-A-18)

### Synthesis of benzyl 1-((S)-2-((tert-butoxycarbonyl)amino)propyl)pyrrolidine-3-carboxylate (IV-2)

Benzyl pyrrolidine-3-carboxylate (II-2) (17.2 g, 83.7 mmol) was dissolved in 70 mL of acetonitrile, Compound III-1 (21.9 g, 92.0 mmol) and DIPEA (32.4 g, 251.1 mmol) were added to obtain a mixture, a temperature of the mixture was raised to 80° C. for reaction for 2 h. Thin layer chromatography (V petroleum ether:V ethyl acetate=1:1) was adopted to monitor the reaction, 200 mL of water was added, ethyl acetate (100 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=3:1) to obtain 25.4 g of colorless oil (IV-2), with a yield of 83.8%. ESI-MS [M+H]⁺ 363.2.

### Synthesis of benzyl (S)-1-(2-aminopropyl)-1H-pyrrole-3-carboxylate (V-2)

Compound IV-2 (7.2 g, 19.9 mmol) was dissolved in 30 mL of dichloromethane, 20 mL of trifluoroacetic acid was added to obtain a mixture, the mixture was stirred at the room temperature for 0.5 h, and thin layer chromatography (V dichloromethane:V methanol=15:1) was adopted to monitor the reaction, a solvent therein was evaporated and removed under reduced pressure, a saturated aqueous sodium bicarbonate solution was added to adjust a pH to 7-8. Dichloromethane (50 mL×5) was used for extraction, organic phases were combined, and then washed with saturated brine, dried over anhydrous sodium sulfate and filtered to obtain a filtrate, and the filtrate was concentrated to obtain 4.4 g of light yellow oil (V-2), with a yield of 84.6%. ESI-MS [M+H]⁺ 263.2.

### Synthesis of benzyl 1-((S)-2-((6-oxo-5-(trifluoromethyl)-1-((2-(trimethylsilyl)ethoxy)methyl)-1,6-dihydropyridazin-4-yl)amino)propyl)pyrrolidine-3-carboxylate (VII-3)

Compound V-2 (2.4 g, 9.0 mmol) was dissolved in 10 mL of 1,4-dioxane, Compound VI-1 (3.3 g, 10.0 mmol) and DIPEA (3.5 g, 27.0 mmol) were added to obtain a mixture, a temperature of the mixture was raised to 90° C. for reaction for 2 h; and thin layer chromatography (V petroleum ether:V ethyl acetate=1:1) was adopted to monitor the reaction. 40 mL of water was added, ethyl acetate (20 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=3:1) to obtain 4.2 g of colorless oil (VII-3), with a yield of 84.4%. ESI-MS [M+H]⁺ 555.3.

### Synthesis of 1-((S)-2-((6-oxo-5-trifluoromethyl-1-((2-(trimethylsilyl)ethoxy)methyl)-1,6-dihydropyridazin-4-yl)amino)propyl)pyrrolidine-3-carboxylic acid (VIII-3)

Compound VII-3 (4.1 g, 7.4 mmol) was dissolved in 20 mL of anhydrous methanol, 500 mg of palladium carbon was added, after a reaction system was replaced with hydrogen, and the reaction was performed at the room temperature for 30 min; and thin layer chromatography (V dichloromethane:V methanol=15:1) was adopted to monitor the reaction, the mixture was subjected to suction filtration to obtain a filter cake, and the filter cake was washed with 10 mL of methanol, and dried to obtain 3.2 g of white solid (VIII-3), with a yield of 93.3%. ESI-MS [M+H]⁺ 465.2.

Synthesis of 4-(trifluoromethyl)-5-(((S)-1-((S)-3-(4-(5-(trifluoromethyl)pyrimidin-2-yl)piperazine-1-carbonyl)pyrrolidin-1-yl)propan-2-yl)amino)-2-((2-(trimethylsilyl)ethoxy)methyl)pyridazin-3(2H)-one (X-18)

Compound VIII-3 (139.2 mg, 0.3 mmol) and Compound IX-1 (70.0 mg, 0.3 mmol) were used as raw materials, and the preparation process was similar to that of Compound X-1 to obtain 129.0 mg of a white solid (X-18), with a yield of 63.5%. ESI-MS: [M+H]⁺ 679.3.

### Synthesis of 4-(trifluoromethyl)-5-(((2S)-1-(3-(4-(5-(trifluoromethyl)pyrimidin-2-yl)piperazine-1-carbonyl)pyrrolidin-1-yl)propan-2-yl)amino)pyridazin-3(2H)-one (I-A-18)

Compound X-18 (88.0 mg, 0.13 mmol) was used as raw material, and the preparation process was similar to that of Compound I-A-1 to obtain 50.0 mg of a white solid (I-A-18), with a yield of 70.4%. ESI-MS [M+H]⁺ 549.2; ¹HNMR (300 MHz, DMSO-d₆) 12.32 (s, 1H), 8.71 (s, 2H), 7.53 (s, 1H), 6.19 (t, J=1.8 Hz, 1H), 4.50-4.40 (m, 1H), 4.10-4.05 (m, 2H), 3.81-3.70 (m, 4H), 3.67-3.60 (m, 4H), 1.92-1.22 (m, 11H), 1.22 (d, J=6.3 Hz, 3H).

With reference to the preparation method described in Example 2, the following compounds were prepared:

| Compound | ¹H-NMR | MS |
|---|---|---|
| I-A-19 | ¹H NMR (300 MHz, DMSO-d₆) δ 12.41 (s, 1H), 8.73 (s, 2H), | ESI-MS: |
| | 7.90 (s, 1H), 6.45-6.30 (m, 1H), 4.11-3.98 (m, 1H), 3.90-3.76 (m, | [M + H]⁺ 563.2 |
| | 4H), 3.65-3.50 (m, 4H), 2.84 (t, J = 12 Hz, 2H), 2.59 (d, J = 12 | |
| | Hz, 1H), 2.45-2.35 (m, 1H), 2.10 (dt, J₁ = 39 Hz, J₂ = 12 Hz, 2H), | |
| | 1.65-1.40 (m, 4H), 1.26-1.22 (m, 1H), 1.16 (d, J = 6.3 Hz, 3H). | |
| I-A-20 | ¹H NMR (400 MHz, DMSO-d₆) δ 12.48 (s, 1H), 8.72 (s, 2H), | ESI-MS: |
| | 7.90 (s, 1H), 6.90-6.80 (m, 1H), 3.85-3.75 (m, 4H), 3.60-3.45 (m, | [M + H]⁺ 520.2 |
| | 6H), 3.35-3.30 (m, 3H), 3.23-3.13 (m, 2H), 2.57-2.52 (m, 2H). | |
| I-A-21 | ¹H NMR (400 MHz, DMSO-d₆) δ 12.45 (s, 1H), 8.76 (s, 2H), | ESI-MS: |
| | 7.90 (s, 1H), 6.49-6.35 (m, 1H), 4.10-3.90 (m, 1H), 3.92-3.72 (m, | [M + H]⁺ 549.2 |
| | 4H), 3.62-3.42 (m, 13H), 1.13 (d, J = 6.3 Hz, 3H). | |
| I-A-22 | ¹H NMR (300 MHz, DMSO-d₆) δ 12.42 (s, 1H), 8.34 (s, 2H), | ESI-MS: |
| | 7.90 (s, 1H), 6.49-6.35 (m, 1H), 4.10-3.90 (m, 1H), 3.92-3.72 (m, | [M + H]⁺ 501.2 |
| | 4H), 3.62-3.42 (m, 11H), 1.12 (d, J = 6.3 Hz, 3H). | |
| I-A-23 | ¹H NMR (300 MHz, DMSO-d₆) δ 12.46 (s, 1H), 8.45 (s, 2H), | ESI-MS: |
| | 7.91 (s, 1H), 6.44-6.35 (m, 1H), 4.03-3.90 (m, 1H), 3.87-3.72 (m, | [M + H]⁺ 545.1 |
| | 4H), 3.57-3.42 (m, 11H), 1.14 (d, J = 6.3 Hz, 3H). | |
| I-A-24 | ¹H NMR (400 MHz, DMSO-d₆) δ 12.53 (s, 1H), 8.72 (s, 2H), | ESI-MS: |
| | 8.00 (s, 1H), 7.16 (s, 1H), 4.00 (m, 1H), 3.83-3.74 (m, 4H), 3.66- | [M + H]⁺ 547.2 |
| | 3.45 (m, 6H), 3.28-3.13 (m, 4H), 1.24 (t, J = 7.2 Hz, 1H), 0.61- | |
| | 0.52 (m, 4H). | |

### Example 3: Synthesis of (S)-5-((1-(3-(4-(2,2-difluorobenzo[d][1,3]dioxol-5-yl)piperazine-1-carbonyl)azetidin-1-yl)propan-2-yl)amino)-4-(trifluoromethyl)pyridazin-3(2H)-one (I-B-1)

Compound X-25 (113.0 mg, 0.17 mmol) was used as raw material, and the preparation process was similar to that of Compound I-A-1 to obtain 80.0 mg of a white solid (I-B-1), with a yield of 86.5%. ESI-MS [M+H]⁺ 545.2; ¹H NMR (400 MHz, DMSO-d₆) δ 12.46 (s, 1H), 7.89 (s, 1H), 7.22 (d, J=8.8 Hz, 1H), 7.09 (d, J=2.4 Hz, 1H), 6.70 (dd, J₁=8.8 Hz, J₂=2.4 Hz, 1H), 6.45-6.35 (m, 1H), 4.00-3.90 (m, 1H), 3.65-3.45 (m, 4H), 3.45-3.35 (m, 3H), 3.26-3.18 (m, 2H), 3.10-3.00 (m, 4H), 2.65-2.54 (m, 2H), 1.12 (d, J=6.3 Hz, 3H).

### Example 4: Synthesis of (S)-4-(trifluoromethyl)-5-((1-(3-(4-(5-(trifluoromethyl)pyrimidin-2-yl)piperazine-1-carbonyl)-1H-pyrrol-1-yl)propan-2-yl)amino)pyridazin-3(2H)-one (I-C-1)

### Synthesis of (S)-1-(2-tert-butyloxycarbonylamino)propyl)-1H-pyrrol-3-methyl formate (XIV-1)

1H-pyrrol-3-methyl formate (XIII-1) (1.3 g, 10.3 mmol) was dissolved in 5 mL of anhydrous DMF, sodium hydride (0.8 g, 20.0 mmol) was added under ice bath to obtain a mixture, a temperature of the mixture was raised to the room temperature and stirred for 1 h; Compound III-1 (3.6 g, 15.0 mmol) was then added under ice bath to obtain a mixture, and the mixture was then heated to 50° C. and reacted for 6 h. Thin layer chromatography (V petroleum ether:V acetone=8:1) was adopted to monitor the reaction. 10 mL of ice water was added to quench the reaction, ethyl acetate (20 mL×3) was used for extraction, organic phases were combined, and then washed with saturated brine, dried over anhydrous sodium sulfate, filtered and concentrated. A crude product was purified by silica gel column chromatography (V petroleum ether:V acetone=11:1) to obtain 2.4 g of a white solid (XIV-1), with a yield of 82.8%. ESI-MS [M+H]⁺ 283.1; ¹H NMR (300 MHz, CDCl₃) δ 7.23 (t, J=2.1 Hz, 1H), 6.55 (d, J=2.1 Hz, 2H), 4.49 (d, J=6.9 Hz, 1H), 3.92-3.84 (m, 2H), 3.77 (s, 3H), 1.41 (s, 9H), 1.07 (d, J=6.9 Hz, 3H).

### Synthesis of (S)-1-(2-tert-butyl)oxycarbonylamino)propyl)-1H-pyrrol-3-carboxylic acid (XV-1)

Compound XIV-1 (2.4 g, 8.5 mmol) was dissolved in 20 mL of a mixed solvent of tetrahydrofuran and methanol (V tetrahydrofuran:V methanol=1:1), a solution obtained by dissolving lithium hydroxide monohydrate (1.8 g, 42.5 mmol) in 4 mL of water was added to the above reaction solution, and a temperature was raised to 60° C. for reaction for 12 h. Thin layer chromatography (V petroleum ether:V ethyl acetate=4:1) was adopted to monitor the reaction, 20 mL of water was added, ethyl acetate (20 mL×2) was used for extraction, aqueous phases were collected, and the pH was adjusted to 5-6 with 2 mol/L of diluted hydrochloric acid; and ethyl acetate (20 mL×3) was used for extraction, organic phases were combined, and then washed with saturated brine, dried over anhydrous sodium sulfate, filtered and concentrated to obtain 1.9 g of a white solid (XV-1), with a yield of 83.4%. ESI-MS [M+H]⁺ 269.1; ¹H NMR (300 MHz, DMSO-d) δ 11.65 (s, 1H), 7.30 (t, J=2.1 Hz, 1H), 6.86 (d, J=10.8 Hz, 1H), 6.71 (t, J=2.4 Hz, 1H), 6.32 (t, J=2.1 Hz, 1H), 3.96-3.66 (m, 3H), 1.33 (s, 9H), 0.98 (d, J=6.6 Hz, 3H).

### Synthesis of (S)-(1-(3-(4-(5-(trifluoromethyl)pyrimidin-2-yl)piperazine-1-carbonyl)-1H-pyrrol-1-yl)propan-2-yl)tert-butyl carbamate (XVI-1)

Compound XV-1 (1.0 g, 3.9 mmol) was dissolved in 6 mL of dichloromethane, HATU (1.6 g, 4.3 mmol) was added to obtain a mixture, and the mixture was stirred at the room temperature for 15 min; Compound IX-1 (1.0 g, 4.3 mmol) and DIPEA (1.0 g, 7.80 mmol) were then added in sequence to obtain a mixture, and the mixture was reacted at the room temperature for 30 min. Thin layer chromatography (V petroleum ether:V ethyl acetate=1:1) was adopted to monitor the reaction, 20 mL of water was added, ethyl acetate (10 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=3:1) to obtain 1.4 g of a white solid (XVI-1), with a yield of 74.5%. ESI-MS [M+H]⁺ 483.2; ¹H NMR (300 MHz, DMSO-d₆) δ 8.71 (s, 2H), 7.14 (t, J=2.1 Hz, 1H), 6.90 (d, J=10.8 Hz, 1H), 6.73 (t, J=2.4 Hz, 1H), 6.25 (t, J=2.1 Hz, 1H), 3.94-3.66 (m, 11H), 1.33 (s, 9H), 0.98 (d, J=6.6 Hz, 3H).

### Synthesis of (S)-(1-(3-(4-(5-(trifluoromethyl)pyrimidin-2-yl)piperazine-1-carbonyl)-1H-pyrrol-1-yl)propan-2-yl)amino (XVII-1)

Compound XVI-1 (1.4 g, 2.9 mmol) was dissolved in 5 mL of dichloromethane, 3 mL of trifluoroacetic acid was added, and the reaction was performed at the room temperature for 15 min; thin layer chromatography (V dichloromethane:V methanol=5:1) was adopted to monitor the reaction, a solvent therein was evaporated and removed under reduced pressure, saturated aqueous sodium bicarbonate solution was added to adjust the pH of a system to 7-8, dichloromethane (20 mL×5) was used for extraction, organic phases were combined, and then washed with saturated brine, dried over anhydrous sodium sulfate, filtered and concentrated to obtain 0.9 g of a light yellow solid (XVII-1), with a yield of 90.9%. ESI-MS [M+H]⁺ 383.2; ¹H NMR (300 MHz, DMSO-d₆) δ 8.74 (s, 2H), 7.21 (t, J=2.1 Hz, 1H), 6.81 (t, J=2.4 Hz, 1H), 6.27 (t, J=2.1 Hz, 1H), 3.94-3.66 (m, 11H), 0.98 (d, J=6.6 Hz, 3H).

Synthesis of (S)-4-trifluoromethyl-5-((1-(3-(4-(5-trifluoromethyl)pyrimidin-2-yl) piperazine-1-carbonyl)-1H-pyrrol-1-yl)propan-2-yl)amino)-2-((2-trimethylsilylethoxymethyl)pyridazin-3(2H)-one (XVIII-1)

Compound VI-1 (1.0 g, 3.2 mmol) was dissolved in 6 mL of 1,4-dioxane, Compound XVII-1 (1.1 g, 2.9 mmol) and DIPEA (1.1 g, 8.6 mmol) were added to obtain a mixture, a temperature of the mixture was raised to 90° C. for reaction for 2 h; and thin layer chromatography (V petroleum ether:V ethyl acetate=1:1) was adopted to monitor the reaction, 20 mL of water was added, ethyl acetate (10 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=3:1) to obtain 1.5 g of a white solid (XVIII-1), with a yield of 69.4%. ESI-MS [M+H]⁺ 675.2; ¹H NMR (300 MHz, DMSO-d₆) δ 8.74 (s, 2H), 7.63 (s, 1H), 7.14 (t, J=2.1 Hz, 1H), 6.75 (t, J=2.7 Hz, 1H), 6.73-6.66 (m, 1H), 6.21-6.17 (m, 1H), 5.18-5.10 (m, 2H), 4.50-4.40 (m, 1H), 4.10-4.05 (m, 2H), 3.88-3.80 (m, 4H), 3.67-3.60 (m, 4H), 3.53 (t, J=8.1 Hz, 2H), 1.24 (d, J=6.3 Hz, 3H), 1.18 (t, J=7.2 Hz, 1H), 0.81 (t, J=8.1 Hz, 2H), −0.06 (s, 9H).

### (S)-4-(trifluoromethyl)-5-((1-(3-(4-(5-(trifluoromethyl)pyrimidin-2-yl)piperazine-1-carbonyl)-1H-pyrrol-1-yl)propan-2-yl)amino)pyridazin-3(2H)-one (I-C-1)

Compound XVIII-1 (674.0 mg, 1.0 mmol) was dissolved in 5 mL of dichloromethane, 4 mL of trifluoroacetic acid was added, and the reaction was performed at the room temperature for 3 h; and thin layer chromatography (V dichloromethane:V methanol=1:4) was adopted to monitor the reaction, a solvent therein was evaporated and removed under reduced pressure, saturated aqueous sodium bicarbonate solution was added to adjust a pH value thereof to 7-8, 10 mL of water was added, dichloromethane (10 mL×3) was used for extraction, organic phases were combined, and then washed with saturated brine, dried over anhydrous sodium sulfate, filtered and concentrated. A crude product was separated and purified by silica gel column chromatography (V petroleum ether:V ethyl acetate=1:1) to obtain 350.0 mg of a white solid (I-C-1), with a yield of 64.3%. ESI-MS [M+H]⁺ 544.2; ¹H NMR (300 MHz, DMSO-d₆) δ 12.37 (s, 1H), 8.73 (s, 2H), 7.90 (s, 1H), 7.14 (t, J=1.5 Hz, 1H), 6.74 (t, J=2.4 Hz, 1H), 6.54-6.45 (m, 1H), 6.19 (t, J=1.8 Hz, 1H), 4.50-4.40 (m, 1H), 4.10-4.05 (m, 2H), 3.88-3.80 (m, 4H), 3.67-3.60 (m, 4H), 1.22 (d, J=6.3 Hz, 3H).

### Example 5: Synthesis of 4-(trifluoromethyl)-5-((1-(1-(1-(5-(trifluoromethyl)pyrimidin-2-yl)piperidine-4-carbonyl)azetidin-3-yl)propan-2-yl)amino)pyridazin-3(2H)-one (I-D-1)

### Synthesis of 2-(1-(1-(5-trifluoromethylpyrimidine-2-yl) piperidine-4-carbonyl)azetidine-3-yl)methyl acetate (XXI-1)

1-(5-(trifluoromethyl)pyrimidin-2-yl)piperidine-4-carboxylic acid (XIX-1) (1.1 g, 4.0 mmol) was dissolved in 5 mL of dichloromethane, HATU (1.7 g, 4.4 mmol) was added to obtain a mixture, and the mixture was stirred at the room temperature for 15 min; 2-(azetidin-3-yl)methyl acetate (XX-1) (0.5 g, 4.0 mmol) and DIPEA (1.0 g, 8.0 mmol) were added, and the reaction was then performed at the room temperature for 30 min; and thin layer chromatography (V petroleum ether:V ethyl acetate=3:1) was adopted to monitor the reaction, 10 mL of water was added, dichloromethane (5 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=1:1) to obtain 1.5 g of a white solid (XXI-1), with a yield of 97.1%. ESI-MS [M+H]⁺ 387.2; ¹HNMR (300 MHz, CDCl₃) δ 8.45 (s, 2H), 4.88-4.78 (m, 2H), 4.38 (t, J=8.4 Hz, 1H), 4.16 (t, J=9.6 Hz, 1H), 3.95-3.87 (m, 1H), 3.69 (s, 3H), 3.06-2.95 (m, 3H), 2.70-2.64 (m, 2H), 2.50-2.40 (m, 1H), 1.80-1.70 (m, 4H).

### Synthesis of 2-(1-(1-(5-(trifluoromethyl)pyrimidin-2-yl) piperidine-4-carbonyl)azetidin-3-yl)acetic acid (XXII-1)

Compound XXI-1 (1.5 g, 3.8 mmol) was dissolved in 5 mL of tetrahydrofuran, and 5 mL of methanol was added, followed by 2 mL of 5 mol/L aqueous sodium hydroxide solution, and the reaction was then performed at the room temperature for 1 h; and thin layer chromatography (V petroleum ether:V ethyl acetate=1:1) was adopted to monitor the reaction. 20 mL of water was added, and ethyl acetate (10 mL×2) was used for extraction. The pH of an aqueous layer was adjusted to 4 with diluted hydrochloric acid, a white solid was precipitated and then filtered to obtain a filter cake, the filter cake was washed with 5 mL of water, the filter cake was collected and dried under vacuum to obtain 1.2 g of a white solid (XXII-1), with a yield of 83.5%. ESI-MS [M+H]⁺ 373.2; ¹HNMR (300 MHz, CDCl₃) δ 8.45 (s, 2H), 4.82 (dt, J₁=13.5 Hz, J₂=3.9 Hz, 2H), 4.38 (t, J=8.4 Hz, 1H), 4.16 (t, J=9.6 Hz, 1H), 3.95-3.87 (m, 1H), 3.06-2.95 (m, 3H), 2.70-2.64 (m, 2H), 2.50-2.40 (m, 1H), 1.80-1.70 (m, 4H).

### Synthesis of N-methoxyl-N-methyl-2-(1-(1-(5-(trifluoromethyl)pyrimidin-2-yl) piperidine-4-carbonyl)azetidin-3-yl)acetamide (XXIII-1)

Compound XXII-1 (1.5 g, 4.0 mmol) was dissolved in 6 mL of dichloromethane, CDI (1.0 g, 6.0 mmol) and dimethylhydroxylamine hydrochloride (1.2 g, 12.0 mmol), and the reaction was then performed at the room temperature for 1 h; and thin layer chromatography (V dichloromethane:V methanol=15:1) was adopted to monitor the reaction, 10 mL of water was added, dichloromethane (8 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=1:1) to obtain 1.2 g of a white solid (XXIII-1), with a yield of 69.9%. ESI-MS [M+H]⁺ 416.2; ¹HNMR (300 MHz, CDCl₃) δ 8.45 (s, 2H), 4.88-4.78 (m, 2H), 4.38 (t, J=8.4 Hz, 1H), 4.16 (t, J=9.6 Hz, 1H), 3.95-3.87 (m, 1H), 3.69 (s, 3H), 3.06-2.95 (m, 3H), 2.72 (s, 3H), 2.70-2.64 (m, 2H), 2.50-2.40 (m, 1H), 1.80-1.70 (m, 4H).

### Synthesis of 1-(1-(1-(5-(trifluoromethyl)pyrimidin-2-yl)piperidine-4-carbonyl)azetidin-3-yl)propan-2-one (XXIV-1)

Compound XXIII-1 (0.8 g, 2.0 mmol) was dissolved in 5 mL of anhydrous tetrahydrofuran, and methylmagnesium bromide (1 mol/L, 4.0 mL) was added at −15° C. to obtain a mixture. After the addition, the mixture was kept warm for reaction for 4 h, and thin layer chromatography (V dichloromethane:V methanol=15:1) was adopted to monitor the reaction. The reaction was quenched by adding 10 mL of water, stirred at the room temperature for 15 min, ethyl acetate (8 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=1:1) to obtain 450.0 mg of a white solid (XXIV-1), with a yield of 60.8%. ESI-MS [M+H]⁺ 371.2; ¹HNMR (300 MHz, CDCl₃) δ 8.45 (s, 2H), 4.88-4.78 (m, 2H), 4.38 (t, J=8.4 Hz, 1H), 4.16 (t, J=9.6 Hz, 1H), 3.95-3.87 (m, 1H), 3.06-2.95 (m, 3H), 2.70-2.64 (m, 2H), 2.50-2.40 (m, 1H), 2.20 (s, 3H), 1.80-1.70 (m, 4H).

### Synthesis of (3-(2-aminopropyl)azetidin-1-yl)(1-(5-(trifluoromethyl)pyrimidin-2-yl)piperidin-4-yl)methanone (XXV-1)

Compound XXIV-1 (0.5 g, 1.4 mmol) was dissolved in 5 mL of methanol, and ammonium formate (0.4 g, 7.0 mmol) and dichloro(pentamethylcyclopentadienyl)rhodium (III) dimer (43.0 mg, 0.07 mmol) were added in sequence to obtain a mixture, and a temperature of the mixture was raised to 70° C. for reaction for 3 h; and thin layer chromatography (V dichloromethane:V methanol=10:1) was adopted to monitor the reaction, a solvent therein was evaporated and removed under reduced pressure, 2 mol/L of hydrochloric acid was added to adjust a pH value thereof to 1-2 to obtain a mixture, and the mixture was stirred at the room temperature for 10 min. Ethyl acetate (5 mL×2) was used for extraction, aqueous layers were adjusted to pH 8-9 with 5 mol/L of aqueous sodium hydroxide solution, and ethyl acetate (10 mL×5) was used for extraction again, organic phases were combined, and then washed with saturated brine, dried over anhydrous sodium sulfate, filtered and concentrated. A crude product was purified by silica gel column chromatography (V dichloromethane:V methanol=10:1) to obtain 370.0 mg of a white solid (XXV-1), with a yield of 71.3%. ESI-MS [M+H]⁺ 372.2; ¹HNMR (300 MHz, DMSO-d₆) δ 8.70 (s, 2H), 4.79-4.69 (m, 2H), 4.34 (t, J=8.4 Hz, 1H), 4.01-3.91 (m, 5H), 3.55-3.45 (m, 1H), 3.15-2.95 (m, 3H), 2.78-2.68 (m, 1H), 1.80-1.65 (m, 3H), 1.51-1.36 (m, 2H), 1.11 (d, J=6.3 Hz, 3H).

### Synthesis of 4-(trifluoromethyl)-5-((1-(1-(1-(5-(trifluoromethyl)pyrimidin-2-yl)piperidine-4-carbonyl)azetidin-3-yl)propan-2-yl)amino)-2-((2-(trimethylsilyl)ethoxy)methyl)pyridazin-3(2H)-one (XXVI-1)

Compound XXV-1 (372.0 mg, 1.0 mmol) was dissolved in 4 mL of 1,4-dioxane, Compound VI-1 (350.0 mg, 1.1 mmol) was added to obtain a mixture, DIPEA (388.0 mg, 3.0 mmol) was then added to obtain a mixture, a temperature of the mixture was raised to 90° C. for reaction for 3 h; and thin layer chromatography (V dichloromethane:V methanol=15:1) was adopted to monitor the reaction. The mixture was cooled down to the room temperature, 10 mL of water was added, ethyl acetate (8 mL×3) was used for extraction, organic phases were combined, and then washed with saturated brine, dried over anhydrous sodium sulfate, filtered and concentrated. A crude product was purified by silica gel column chromatography (V dichloromethane:Vmethanol=70:1) to obtain 370.0 mg of a white solid (XXVI-1), with a yield of 56.7%. ESI-MS [M+H]⁺ 654.3; ¹H NMR (300 MHz, DMSO-d₆) δ 8.73 (s, 2H), 8.00 (s, 1H), 6.59 (s, 1H), 5.21 (s, 2H), 4.05-3.95 (m, 1H), 3.83-3.74 (m, 4H), 3.66-3.45 (m, 9H), 3.28-3.13 (m, 5H), 1.24 (t, J=7.2 Hz, 1H), 1.18 (d, J=6.3 Hz, 3H), 0.81 (t, J=8.1 Hz, 2H), −0.06 (s, 9H).

### Synthesis of 4-(trifluoromethyl)-5-((1-(1-(1-(5-(trifluoromethyl)pyrimidin-2-yl)piperidine-4-carbonyl)azetidin-3-yl)propan-2-yl)amino)pyridazin-3(2H)-one (I-D-1)

Compound XXVI-1 (327.0 mg, 0.5 mmol) was dissolved in 3 mL of dichloromethane, and 2 mL of trifluoroacetic acid was added to obtain a mixture, the mixture was reacted at the room temperature for 3 h; and thin layer chromatography (V dichloromethane:Vmethanol=15:1) was adopted to monitor the reaction, a solvent therein was evaporated and removed under reduced pressure, saturated aqueous sodium bicarbonate solution was added to adjust a pH value thereof to 7-8, 10 mL of water was added, dichloromethane (10 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=1:1) to obtain 230.0 mg of a white solid (I-D-1), with a yield of 86.1%. ESI-MS [M+H]⁺ 534.2; ¹H NMR (400 MHz, DMSO-d₆) δ 12.48 (s, 1H), 8.67 (s, 2H), 7.92 (s, 1H), 6.43-6.35 (m, 1H), 4.70-4.60 (m, 2H), 4.30-4.20 (m, 1H), 4.05-3.95 (m, 1H), 3.90-3.82 (m, 1H), 3.55-3.46 (m, 1H), 3.10-3.00 (m, 2H), 2.66-2.53 (m, 2H), 2.03-1.95 (m, 1H), 1.82-1.65 (m, 3H), 1.50-1.35 (m, 2H), 1.17 (d, J=6.3 Hz, 3H).

### Example 6: Synthesis of N-(4-((6-oxo-5-(trifluoromethyl)-1,6-dihydropyridazin-4-yl)amino)butyl)-4-(5-(trifluoromethyl)pyrimidin-2-yl)piperazine-1-carboxamide (I-E-1)

### Synthesis of 4-nitrophenyl 4-(5-(trifluoromethyl)pyrimidin-2-yl)piperazine-1-carboxylate (XXVIII-1)

Compound IX-1 (464.0 mg, 2.0 mmol) was dissolved in 3 mL of dichloromethane, triethylamine (404.0 mg, 4.0 mmol) was added, Compound XXVII-1 (442.2 mg, 2.2 mmol) was added at 0° C. to obtain a mixture, and the mixture was reacted at the room temperature for 1 h; and thin layer chromatography (V petroleum ether:V ethyl acetate=4:1) was adopted to monitor the reaction, 5 mL of water was added, dichloromethane (5 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=10:1) to obtain 710.0 mg of a yellow solid (XXVIII-1), with a yield of 89.4%. ESI-MS [M+H]⁺ 398.1.

### Synthesis of tert-butyl (4-(4-(5-(trifluoromethyl)pyrimidin-2-yl)piperazine-1-carboxamido)butyl)carbamate (XXX-1)

Compound XXVIII-1 (397.3 mg, 1.0 mmol) was dissolved in 4 mL of 1,4-dioxane, Compound XXIX-1 (207.0 mg, 1.1 mmol) was added, potassium carbonate (414.0 mg, 3.0 mmol) was then added to obtain a mixture, a temperature of the mixture was raised to 90° C. for reaction for 7 h; and thin layer chromatography (V dichloromethane:V methanol=15:1) was adopted to monitor the reaction. The mixture was cooled down to the room temperature, 5 mL of water was added, ethyl acetate (5 mL×3) was used for extraction, organic phases were combined, and then washed with saturated brine, dried over anhydrous sodium sulfate, filtered and concentrated. A crude product was purified by silica gel column chromatography (pure EA) to obtain 350.0 mg of a white solid (XXX-1), with a yield of 78.0%. ESI-MS [M+H]⁺ 447.2.

### Synthesis of N-(4-aminobutyl)-4-(5-(trifluoromethyl)pyrimidin-2-yl)piperazine-1-carboxamide (XXXI-1)

Compound XXXI-1 (223.0 mg, 0.5 mmol) was dissolved in 3 mL of dichloromethane, 3 mL of trifluoroacetic acid was added to obtain a mixture, and the mixture was reacted at the room temperature for 0.5 h; thin layer chromatography (V petroleum ether:V ethyl acetate=1:4) was adopted to monitor the reaction, a solvent therein was evaporated and removed under reduced pressure, saturated aqueous sodium bicarbonate solution was added to adjust a pH value thereof to 7-8, 4 mL of water was added, dichloromethane (5 mL×7) was used for extraction, organic phases were combined, and then washed with saturated brine, dried over anhydrous sodium sulfate, filtered and concentrated to obtain a filtrate, and the filtrate was concentrated to obtain 120.0 mg of a white solid (XXXI-1) with a yield of 69.4%. ESI-MS [M+H]⁺ 347.2.

### Synthesis of N-(4-((6-oxo-5-(trifluoromethyl)-1-((2-(trimethylsilyl)ethoxy)methyl)-1,6-dihydropyridazin-4-yl)amino)butyl)-4-(5-(trifluoromethyl)pyrimidin-2-yl)piperazine-1-carboxamide (XXXII-1)

Compound XXXI-1 (346.4 mg, 1.0 mmol) was dissolved in 4 mL of 1,4-dioxane, Compound VI-1 (350.0 mg, 1.1 mmol) and DIPEA (388.0 mg, 3.0 mmol) were added to obtain a mixture, a temperature of the mixture was raised to 90° C. for reaction for 3 h; and thin layer chromatography (V dichloromethane:V methanol=15:1) was adopted to monitor the reaction. The mixture was cooled down to the room temperature, 10 mL of water was added, ethyl acetate (8 mL×3) was used for extraction, organic phases were combined, and then washed with saturated brine, dried over anhydrous sodium sulfate, filtered and concentrated. A crude product was purified by silica gel column chromatography (V dichloromethane:V methanol=70:1) to obtain 490.0 mg of a white solid (XXXII-1), with a yield of 76.8%. ESI-MS [M+H]⁺ 639.3.

### Synthesis of N-(4-((6-oxo-5-(trifluoromethyl)-1,6-dihydro-pyridazin-4-yl)amino)butyl)-4-(5-(trifluoromethyl)pyrimidin-2-yl)piperazine-1-carboxamide

Compound XXII-1 (314.0 mg, 0.5 mmol) was dissolved in 3 mL of dichloromethane, 2 mL of trifluoroacetic acid was added to obtain a mixture, and the mixture was reacted at the room temperature for 3 h; and thin layer chromatography (V dichloromethane:Vmethanol=15:1) was adopted to monitor the reaction, a solvent therein was evaporated and removed under reduced pressure, saturated aqueous sodium bicarbonate solution was added to adjust a pH value thereof to 7-8, 10 mL of water was added, dichloromethane (10 mL×3) was used for extraction, organic phases were combined, and then washed with saturated brine, dried over anhydrous sodium sulfate, filtered and concentrated. A crude product was purified by silica gel column chromatography (V petroleum ether:V ethyl acetate=1:1) to obtain 210.0 mg of a white solid (I-E-1), with a yield of 82.7%. ESI-MS [M+H]⁺ 509.2; ¹H NMR (300 MHz, DMSO) δ 12.43 (s, 1H), 8.71 (s, 2H), 7.87 (s, 1H), 7.10 (s, 1H), 6.64 (s, 1H), 3.90-3.70 (m, 4H), 3.55-3.41 (m, 6H), 3.12-3.02 (m, 2H), 1.60-1.40 (m, 4H).

With reference to the preparation method described in Example 6, the following compounds were prepared:

| Compound | ¹H-NMR | MS |
|---|---|---|
| I-E-2 | ¹H NMR (300 MHz, DMSO-d₆) δ 12.47 (s, 1H), 8.71 (s, 2H), | ESI-MS: |
| | 7.70 (s, 1H), 7.32 (s, 2H), 6.82 (s, 1H), 5.60-5.40 (m, 2H), 4.41- | [M + H]⁺ 507.2 |
| | 3.74 (m, 12H). | |
| I-E-3 | ¹H NMR (300 MHz, DMSO-d₆) δ 12.44 (s, 1H), 8.71 (s, 2H), | ESI-MS: |
| | 7.87 (s, 1H), 7.21 (s, 1H), 6.70 (t, J = 5.6 Hz, 1H), 3.86-3.77 | [M + H]⁺ 495.2 |
| | (m, 4H), 3.43-3.40 (m, 4H), 3.30-3.22 (m, 2H), 3.21-3.02 (m, | |
| | 2H), 1.74-1.56 (m, 2H). | |
| I-E-4 | ¹H NMR (300 MHz, DMSO-d₆) δ 12.42 (s, 1H), 8.72 (s, 2H), | ESI-MS: |
| | 7.94 (s, 1H), 7.24 (s, 1H), 6.93 (t, J = 5.4 Hz, 1H), 3.8-3.75 (m, | [M + H]⁺ 481.2 |
| | 4H), 3.45-3.39 (m, 4H), 3.39-3.36 (m, 2H), 3.27-3.17 (m, 2H). | |
| I-E-5 | ¹H NMR (300 MHz, DMSO-d₆) δ 12.45 (s, 1H), 8.42-8.32 (m, | ESI-MS: |
| | 2H), 7.87 (s, 1H), 7.23 (s, 1H), 6.72-6.58 (m, 1H), 3.76-3.65 (m, | [M + H]⁺ 427.2 |
| | 4H), 3.64-3.54 (m, 2H), 3.39-3.38 (m, 2H), 3.19-3.04 (m, 4H), | |
| | 1.67-1.57 (m, 2H). | |
| I-E-6 | ¹H NMR (300 MHz, DMSO-d₆) δ 12.42 (s, 1H), 8.37 (d, J = 4.7 | ESI-MS: |
| | Hz, 2H), 7.94 (s, 1H), 7.25 (s, 1H), 6.90 (t, J = 5.4 Hz, 1H), 6.65 | [M + H]⁺ 413.2 |
| | (t, J = 4.7 Hz, 1H), 3.72-3.65 (m, 4H), 3.45-3.36 (m, 6H), 3.25- | |
| | 3.16 (m, 2H). | |
| I-E-7 | ¹H NMR (300 MHz, DMSO-d₆) δ 12.49 (s, 1H), 8.73 (s, 2H), | ESI-MS: |
| | 8.69 (s, 1H), 7.76-7.66 (m, 1H), 7.62 (s, 1H), 7.44 (s, 1H), 7.37 | [M + H]⁺ 543.2 |
| | (d, J = 8.2 Hz, 1H), 7.23 (t, J = 7.8 Hz, 1H), 6.88 (d, J = 7.7 Hz, | |
| | 1H), 4.57 (d, J = 6.1 Hz, 2H), 3.94-3.80 (m, 4H), 3.63-3.47 (m, | |
| | 4H). | |

### Example 7: Inhibitory Activity of Compounds on PARP7 Enzyme

Experimental materials: PARP7 Chemiluminescent Assay Kit, BPS Bioscience; DMSO, China National Pharmaceutical Group, Nivo, PerkinElmer.

### Experimental Method:

### (1) Preparation of Solutions and Buffers:

Preparation of 10×PBS: 720 mg of KH₂PO₄, 45 g of NaCl and 5.311 g of Na₂HPO₄·12H₂O were weighed respectively and dissolved in 500 mL of deionized water, a system pH value was adjusted to 7.4, sterilization was performed at 121° C. for 30 min, and the system after cooling was placed at 4° C. for later use.

Preparation of 1×PBS: 10×PBS was diluted 10 times with deionized water, that is, 9 parts of deionized water was added to 1 part of 10×PBS for dilution.

Preparation of wash buffer: 1×PBS contained 0.05% Tween-20.

Preparation of 1×PARP buffer (prepared freshly before use): 10×PARP buffer was diluted 10 times with deionized water and placed on ice for later use.

### (2) Preparation of Compound Working Solution Concentration:

According to test requirements, a compound to be tested is diluted with 100% DMSO to a desired concentration, and then diluted 10 times with 1×PARP buffer to prepare a 10× compound working solution.

### (3) Experimental Steps:

a. 5× histone mixture was thawed on ice one day prior to the experiment;

b. preparation of 1× histone mixture: 1× histone mixture was prepared through the 5× histone mixture by using 1×PBS; and 25 μL of 1× histone mixture was taken from each well and transferred into a test well plate and incubated overnight at 4° C.;

c. 100 μL of Blocking buffer was taken from each well and added to the test well plate and incubated for 90 min at 25° C.;

d. after the incubation was completed, the liquid in the test well plate was spin-dried, and the test well plate was washed repeatedly for 3 times;

e. 2.5 μL of the compound working solution was taken from each well and added to the test well plate according to an experimental layout diagram; and 1×PARP buffer with a corresponding volume of 10% DMSO was added to positive control wells (Positive Control), and 1×PARP buffer of a corresponding volume was added to blank control wells (Blank);

f. after the enzyme was completely dissolved, an enzyme stock solution was diluted with 1×PARP buffer to 6 ng/μL;

g. 10 μL of enzyme solution was taken from each well and added to the test well plate, the blank control wells were added with the corresponding volume of 1×PARP buffer, and in which case, the enzyme amount was 60 ng per well. Note: this step needs to be performed on ice;

h. 12.5 μL of master mixture was added to each well of the test well plate (12.5 μL master mixture included 1.25 μL of 10×PARP buffer, 1.25 μL of Opti-PARP 10× Assay mixture and 10 μL of water); the test well plate was sealed and incubated at 25° C. for 60 min;

i. after the incubation was completed, the liquid in the test well plate was spin-dried, and the test well plate was washed repeatedly for 3 times;

j. Streptavidin-HRP in a kit was diluted 50 times with a Blocking buffer, 25 μL was taken from each well and added to the test well plate, and incubated at 25° C. for 30 min;

k. after the incubation was completed, the liquid in the test well plate was spin-dried, and the test well plate was washed repeatedly for 3 times;

l. ELISA ECL Substrate A and ELISA ECL Substrate B in the kit was mixed to obtain a mixed solution according to a ratio of 1:1, 50 μL of the mixed solution was added to each well of the test well plate, Nivo was used to perform Luminescence detection immediately, and a luminous value (RLU) was read; and

m. calculation of an enzyme rate: Enzyme Activity=(RLU(Sample)−RLU(Blank))/(RLU(Pos.Ctrl)−RLU(Blank))×100%; and enzyme inhibition rate=1−% Enzyme Activity, and specific results were shown in Table 1 below.

TABLE 1
Enzyme Inhibitory Activity Data of Tested Compounds on PARP 7

| Compound No. | 100 nM Inhibition Rate |
|---|---|
| I-A-1 | +++ |
| I-A-2 | ++ |
| I-A-3 | +++ |
| I-A-4 | +++ |
| I-A-5 | +++ |
| I-A-6 | +++ |
| I-A-7 | +++ |
| I-A-8 | ++ |
| I-A-9 | +++ |
| I-A-10 | +++ |
| I-A-11 | ++ |
| I-A-12 | ++ |
| I-A-13 | +++ |
| I-A-14 | ++ |
| I-A-15 | +++ |
| I-A-16 | +++ |
| I-A-17 | +++ |
| I-A-18 | ++ |
| I-A-19 | ++ |
| I-A-20 | +++ |
| I-A-21 | ++ |
| I-A-22 | ++ |
| I-A-23 | ++ |
| I-A-24 | ++ |
| I-B-1 | +++ |
| I-C-1 | +++ |
| I-D-1 | +++ |
| I-E-1 | +++ |
| I-E-2 | +++ |
| I-E-7 | +++ |

Note:
“+++” means IC₅₀ < 0.1 μM; “++” means IC₅₀ ≥ 0.1 μM and < 0.5 μM; and “+” means IC₅₀ ≥ 0.5 μM.

As shown in Table 1, all the tested compounds in the present invention exhibited good enzyme inhibitory activity against PARP7, and enzyme inhibition IC₅₀ values thereof all reached a nanomolar concentration level. Specifically, the enzyme inhibition IC₅₀ values of Compounds I-A-1, I-A-3-1-A-7, I-A-9-1-A-10, I-A-13, I-A-15-1-A-17, I-A-20, I-B-1, I-C-1, I-D-1, I-E-1, I-E-2 and I-E-7 were all lower than 0.1 μM.

### Example 8: Effect of the Compounds on Interferon Release

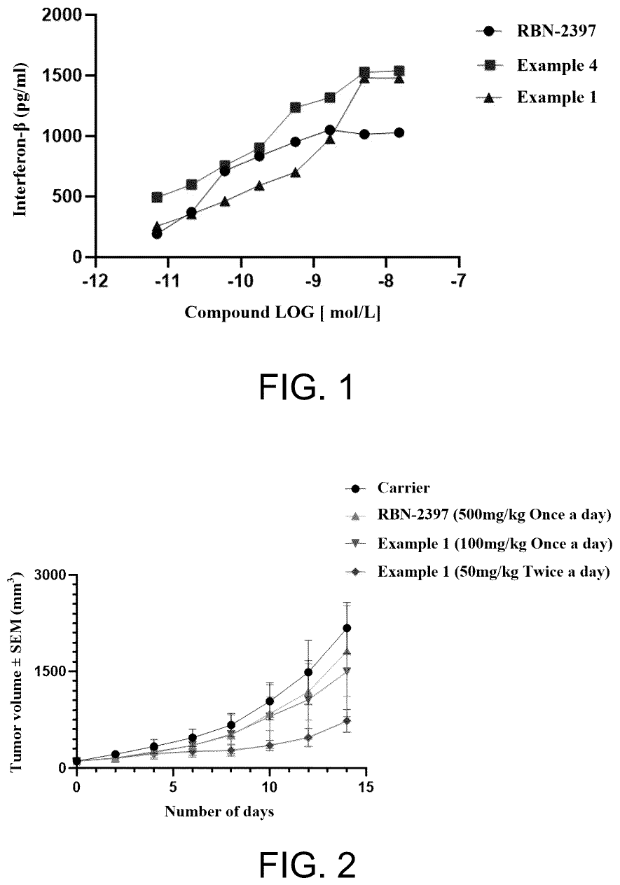

In the presence of STING agonist DMXAA, an interferon-13 level of RAW264.7 cells was induced by a PARP7 inhibitor. The RAW264.7 cells that have grown to a logarithmic growth phase were coated in a 96-well plate, and incubated in a 37° C. and 500 CO₂ incubator overnight until they stuck to walls. The cells were treated with a dose-titrated PARP 7 inhibitor and SO ag/mL of DMXAA for 24 h, and a supernatant therefrom was collected and processed according to kit instructions by ELISA (R&D, Mouse IFN-beta DuoSet Elisa). Results thereof were illustrated in FIG. 1.

As can be seen from FIG. 1, the compounds provided in the present invention could significantly promote the release of interferon β, therefore, they could be used for tumor immunotherapy, and the release amount thereof was superior to that of a positive drug RBN-2397.

### Example 9: Pharmacokinetic Study of Compounds in Rats

Experimental process: 12 male SD rats were selected, 6 of the rats were orally administrated (3 of them were administered with RBN-2397, and the remaining 3 were administered with I-A-1, at a dose of 10 mg/kg), 6 of the rats were administered intravenously (3 of them were administered with RBN-2397, and the remaining 3 were administered with I-A-1, at a dose of 1 mg/kg), blood samples were collected at 0 min, 2 min, 5 min, 10 min, 20 min, 30 min, 60 min, 2 h, 4 h, 6 h, and 8 h, respectively, and then centrifuged (at 3000 rpm for 5 min), a supernatant was collected and analyzed by LC-MS-MS, and results were analyzed by using WinNonlin Software. Specific results were shown in Table 2 below.

TABLE 2
Pharmacokinetic Data of Tested Compounds in SD Rats

| | RBN-2397 | | I-A-1 | |
|---|---|---|---|---|
| | IV | PO | IV | PO |
| | (1 mg/kg) | (10 mg/kg) | (1 mg/kg) | (10 mg/kg) |
| T_{1/2} (h) | 0.28 | 0.75 | 1.72 | 2.57 |
| Cₘₐₓ (ng/mL) | 836.50 | 295.00 | 333.00 | 563.50 |
| AUC (h*ng/mL) | 332.70 | 415.11 | 547.52 | 2564.08 |
| CL (mL/h/kg) | 3033.11 | 22880.52 | 1826.42 | 3777.13 |
| F (%) | | 12.4% | | 46.8% |

IV: represents intravenous injection, and PO: represents intragastric administration.

Experimental results indicated that Compounds I-A-1 provided in the present invention had good pharmacokinetic properties in the SD rats. Compared with the positive drug RBN-2397, Compounds provided in the present invention had a longer half-life, a larger in-vivo exposure, and a better oral bioavailability.

### Example 10: Pharmacokinetic Study of Compounds in Mice

Experimental process: 12 male ICR mice were selected, 6 of the rats were orally administrated (3 of them were administered with RBN-2397, and the remaining 3 were administered with I-A-1, at a dose of 10 mg/kg), 6 of the rats were administered intravenously (3 of them were administered with RBN-2397, and the remaining 3 were administered with I-A-1, at a dose of 1 mg/kg), blood samples were collected at 0 min, 2 min, 5 min, 10 min, 20 min, 30 min, 60 min, 2 h, 4 h, 6 h, and 8 h, respectively, and then centrifuged (at 3000 rpm for 5 min), a supernatant was collected and analyzed by LC-MS-MS, and results were analyzed by using WinNonlin Software. Specific results were shown in Table 3 below.

TABLE 3
Pharmacokinetic Data of Tested Compounds in ICR Mice

| | RBN-2397 | | I-A-1 | |
|---|---|---|---|---|
| | IV | PO | IV | PO |
| | (1 mg/kg) | (10 mg/kg) | (1 mg/kg) | (10 mg/kg) |
| T_{1/2} (h) | 0.15 | 0.90 | 1.44 | 1.74 |
| Cₘₐₓ(ng/mL) | 757.00 | 1306.33 | 418.67 | 2300.00 |
| AUC (h*ng/mL) | 256.00 | 656.00 | 591.10 | 5663.54 |
| CL (mL/h/kg) | 4023.00 | | 1741.46 | |
| F (%) | | 25.67% | | 95.80% |

IV: represents intravenous injection, and PO: represents intragastric administration.

Experimental results indicated that Compounds I-A-1 provided in the present invention had good pharmacokinetic properties in the ICR mice. Compared with the positive drug RBN-2397, Compounds provided in the present invention had a longer half-life and a larger in-vivo exposure, and oral bioavailability reached up to 95.80%.

### Example 11: In Vivo Pharmacodynamic Study of Compounds in Mice

BALB/c mice were subcutaneously inoculated with CT26 cells in the right abdominal skin to develop tumors. Four days after tumor inoculation, 24 mice having a tumor size ranging from 55-75 mm³ (with an average tumor size of 63 mm³) were selected and randomly divided into 4 groups based on their tumor volumes, with 6 mice in each group. Drug administration was started on the day following random grouping (the day of random grouping was identified as Day 0), a carrier (0.5% of methyl cellulose+0.2% of Tween 80), Compound RBN-2397 (500 mg/kg, once a day, intragastric administration for 14 consecutive days), Compound I-A-1 (100 mg/kg, once a day, intragastric administration for 14 consecutive days), Compound I-A-1 (50 mg/kg, twice a day, intragastric administration for 14 consecutive days) were administered respectively, and the tumor size was measured three times per week during an administration period. The entire study was terminated on Day 14, and its efficacy results were shown in FIG. 2.

It can be seen from FIG. 2 that Compound I-A-1 provided in the present invention exhibited significant anti-tumor activity in mice, and compared with the positive drug RBN-2397, Compound I-A-1 provided in the present invention could exert a better anti-tumor effect at a lower dose.

1. A pyridazinone compound, wherein it has a structure as shown in Formula (I), and contains an isomer thereof, a pharmaceutically acceptable salt thereof, or a mixture of the isomer and the pharmaceutically acceptable salt: 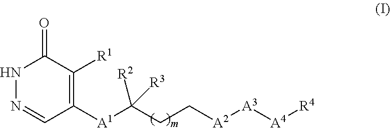
in the formula:
m is selected from 0, 1, 2 or 3;
R¹ is selected from hydrogen, halogen, cyano, trifluoromethyl, C₁-C₆ alkyl, C₁-C₆ alkoxy, methylthio, methanesulfonyl, and carbamoyl;
R² or R³ is independently selected from hydrogen, C₁-C₆ alkyl, substituted C₃-C₆ cycloalkyl or heterocycloalkyl, cyano, or trifluoromethyl; or R² and R³ together with carbon atoms to which they are connected form C₃-C₆ cycloalkyl; a substituent of the C₃-C₆ cycloalkyl is selected from hydrogen, methyl, trifluoromethyl, 2,2-difluoroethyl, methoxy, halogen, cyano, amino, methylamino, dimethylamino, diethylamino, acetyl amino, hydroxyl, acetoxy, carboxyl or methoxycarbonyl, and the substituent is one or more;
R⁴ is selected from substituted aryl, substituted heteroaryl or substituted 1,3-benzodioxanyl, the heteroaryl or the 1,3-benzodioxanyl is selected from hydrogen, halogen, cyano, trifluoromethyl, 2,2-difluoroethyl, C₁-C₆ alkyl, C₃-C₆ cycloalkyl, aryl, C₁-C₆ alkoxy, hydroxy, methoxy, amino, methylamino, dimethylamino, acetyl amino, carboxyl, methanesulfonyl, methoxycarbonyl, or nitro, and a substituent is one or more;
A¹ is selected from —NH—, —O—, —S—, 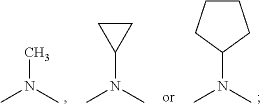
A² is selected from 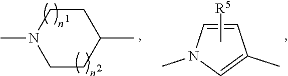
—NH— or —N(CH₃)—, wherein n¹ and n² are independently selected from 0, 1 or 2;
R⁵ is selected from hydrogen, halogen, methyl, trifluoromethyl, cyano, hydroxy, methoxy, amino, methylamino, dimethylamino, diethylamino or acetyl amino; and R⁵ is one or more;
A³ is selected from 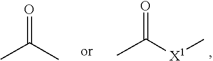
wherein X¹ represents —O—, —NH— or 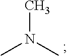
and
A⁴ is selected from 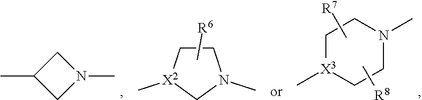
wherein X² and X³ are independently selected from N or CH, R⁶, R⁷ or R⁸ is independently selected from hydrogen, methyl, trifluoromethyl, cyano, hydroxy, methoxy, amino, methylamino, dimethylamino, acetyl amino, carboxyl or methoxycarbonyl.

2. The pyridazinone compound according to claim 1, wherein in the structure:
m is selected from 0, 1 or 2;
R¹ is selected from halogen, cyano or trifluoromethyl;
R² or R³ is independently selected from hydrogen, methyl or trifluoromethyl; and when R² and R³ are different, carbon atoms connected to R² and R³ are in a racemic configuration, an R configuration, or an S configuration;
R⁴ is selected from 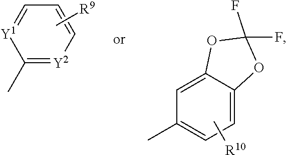
wherein Y¹ or Y² is independently selected from CH or N, R⁹ and R¹⁰ are independently selected from trifluoromethyl, methyl, fluorine, chlorine, bromine, cyano, methoxy, methanesulfonyl, 2,2-difluoroethyl or 4-trifluoromethyl, and R⁹ or R¹⁰ is one or more;
A¹ is selected from —NH—;
A² is selected from 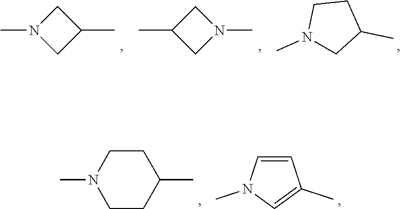
—NH— or —N(CH₃)—;
A³ is selected from 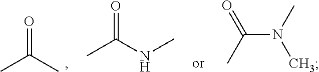
and
A⁴ is selected from

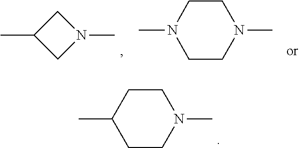

3. The pyridazinone compound according to claim 1, wherein in the structure:
m is 0 or 2.

4. The pyridazinone compound according to claim 1, wherein in the structure:
R¹ is trifluoromethyl; R² is hydrogen; R³ is hydrogen or methyl; and when R³ is methyl, a carbon atom connected to R³ is the S configuration.

5. The pyridazinone compound according to claim 1, wherein in the structure:
A² is selected from 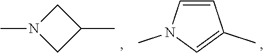
or —NH—; A³ is 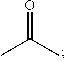
and A⁴ is

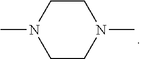

6. The pyridazinone compound according to claim 1, wherein in the structure:
R⁴ is selected from

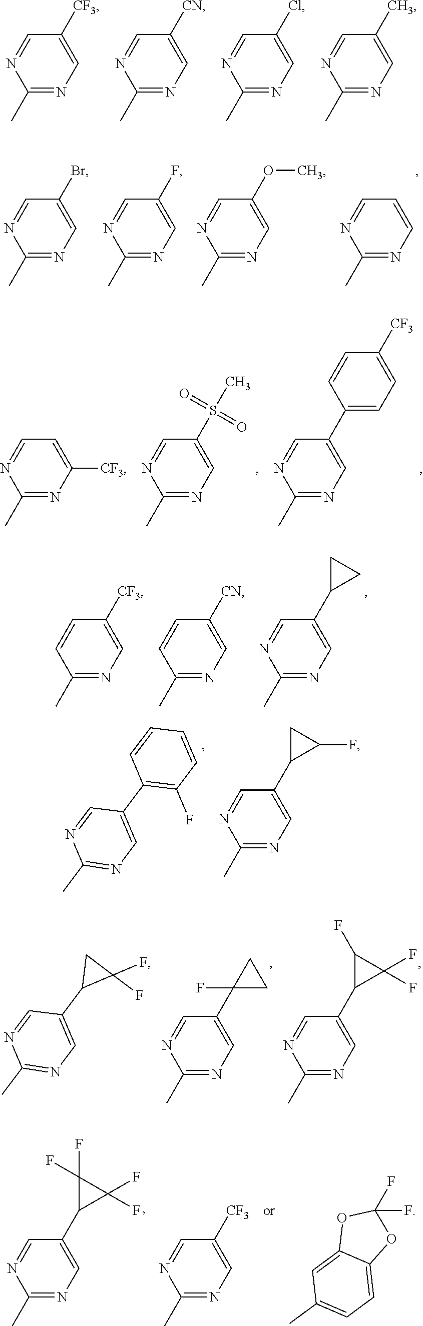

7. The pyridazinone compound according to claim 1, wherein the pyridazinone compound is any of the following compounds:
(S)-4-(trifluoromethyl)-5-((1-(3-(4-(5-(trifluoromethyl)pyrimidin-2-yl)piperazine-1-carbonyl)azetidin-1-yl)propan-2-yl)amino)pyridazin-3(2H)-one (I-A-1);
(S)-4-(trifluoromethyl)-5-((1-(3-(4-(5-(4-(trifluoromethyl)phenyl)pyrimidin-2-yl)piperazine-1-carbonyl)azetidin-1-yl)propan-2-yl)amino)pyridazin-3(2H)-one (I-A-2);
(S)-2-(4-(1-(2-((6-oxo-5-(trifluoromethyl)-1,6-dihydropyridazin-4-yl)amino)propyl)azetidine-3-carbonyl)piperazin-1-yl)pyrimidine-5-carbonitrile (I-A-3);
(S)-5-((1-(3-(4-(5-(methylsulfonyl)pyrimidin-2-yl)piperazine-1-carbonyl)azetidin-1-yl)propan-2-yl)amino)-4-(trifluoromethyl)pyridazin-3(2H)-one (I-A-4);
(S)-5-((1-(3-(4-(5-chloropyrimidin-2-yl)piperazine-1-carbonyl)azetidin-1-yl)propan-2-yl)amino)-4-(trifluoromethyl)pyridazin-3(2H)-one (I-A-5);
(S)-5-((1-(3-(4-(5-methylpyrimidin-2-yl)piperazine-1-carbonyl)azetidin-1-yl)propan-2-yl)amino)-4-(trifluoromethyl)pyridazin-3(2H)-one (I-A-6);
(S)-5-((1-(3-(4-(5-bromopyrimidin-2-yl)piperazine-1-carbonyl)azetidin-1-yl)propan-2-yl)amino)-4-(trifluoromethyl)pyridazin-3(2H)-one (I-A-7);
(S)-5-((1-(3-(4-(5-fluoropyrimidin-2-yl)piperazine-1-carbonyl)azetidin-1-yl)propan-2-yl)amino)-4-(trifluoromethyl)pyridazin-3(2H)-one (I-A-8);
(S)-5-((1-(3-(4-(5-methoxypyrimidin-2-yl)piperazine-1-carbonyl)azetidin-1-yl)propan-2-yl)amino)-4-(trifluoromethyl)pyridazin-3(2H)-one (I-A-9);
(S)-4-(trifluoromethyl)-5-((1-(3-(4-(5-(trifluoromethyl)pyridin-2-yl)piperazine-1-carbonyl)azetidin-1-yl)propan-2-yl)amino)pyridazin-3(2H)-one (I-A-10);
(S)-4-(trifluoromethyl)-5-((1-(3-(4-(4-trifluoromethyl)phenyl)piperazine-1-carbonyl)azetidin-1-yl)propan-2-yl)amino)pyridazin-3(2H)-one (I-A-11);
(S)-5-((1-(3-(4-dibenzo[b,d]furan-4-yl)piperazine-1-carbonyl)azetidin-1-yl)propan-2-yl)amino)-4-(trifluoromethyl)pyridazin-3(2H)-one (I-A-12);
(S)-4-(trifluoromethyl)-5-((1-(3-(4-(4-(trifluoromethyl)pyrimidin-2-yl)piperazine-1-carbonyl)azetidin-1-yl)propan-2-yl)amino)pyridazin-3(2H)-one (I-A-13);
(S)-5-((1-(3-(4-(pyrimidin-2-yl)piperazine-1-carbonyl)azetidin-1-yl)propan-2-yl)amino)-4-(trifluoromethyl)pyridazin-3(2H)-one (I-A-14);
(S)-1-(2-((6-oxo-5-(trifluoromethyl)-1,6-dihydropyridazin-4-yl)amino)propyl)-N-(1-(5-(trifluoromethyl)pyrimidin-2-yl)azetidin-3-yl)azetidine-3-carboxamide (I-A-15);
(S)-N-methyl-1-(2-((6-oxo-5-(trifluoromethyl)-1,6-dihydropyridazin-4-yl)amino)propyl)-N-(1-(5-(trifluoromethyl)pyrimidin-2-yl)azetidin-3-yl)azetidine-3-carboxamide (I-A-16);
5-(((2S)-1-(3-(2,6-dimethyl-4-(5-(trifluoromethyl)pyrimidin-2-yl)piperazine-1-carbonyl)azetidin-1-yl)propan-2-yl)amino)-4-(trifluoromethyl)pyridazin-3(2H)-one (I-A-17);
4-(trifluoromethyl)-5-(((2S)-1-(3-(4-(5-(trifluoromethyl)pyrimidin-2-yl)piperazine-1-carbonyl)pyrrolidin-1-yl)propan-2-yl)amino)pyridazin-3(2H)-one (I-A-18);
(S)-4-(trifluoromethyl)-5-((1-(4-(4-(5-(trifluoromethyl)pyrimidin-2-yl)piperazine-1-carbonyl)piperidin-1-yl)propan-2-yl)amino)pyridazin-3(2H)-one (I-A-19);
4-(trifluoromethyl)-5-((2-(3-(4-(5-(trifluoromethyl)pyrimidin-2-yl)piperazine-1-carbonyl)azetidin-1-yl)ethyl)amino)pyridazin-3(2H)-one (I-A-20);
(S)-4-(trifluoromethyl)-5-((4-(3-(4-(5-(trifluoromethyl)pyrimidin-2-yl)piperazine-1-carbonyl)azetidin-1-yl)butan-2-yl)amino)pyridazin-3(2H)-one (I-A-21);
(S)-4-chloro-5-((1-(3-(4-(5-(trifluoromethyl)pyrimidin-2-yl)piperazine-1-carbonyl)azetidin-1-yl)propan-2-yl)amino)pyridazin-3(2H)-one (I-A-22);
(S)-4-bromo-5-((1-(3-(4-(5-(trifluoromethyl)pyrimidin-2-yl)piperazine-1-carbonyl)azetidin-1-yl)propan-2-yl)amino)pyridazin-3(2H)-one (I-A-23);
4-(trifluoromethyl)-5-((1-((3-(4-(5-(trifluoromethyl)pyrimidin-2-yl)piperazine-1-carbonyl)azetidin-1-yl)methyl)cyclopropyl)amino)pyridazin-3(2H)-one (I-A-24);
(S)-5-((1-(3-(4-(2,2-difluorobenzo[d][1,3]dioxol-5-yl)piperazine-1-carbonyl)azetidin-1-yl)propan-2-yl)amino)-4-(trifluoromethyl)pyridazin-3(2H)-one (I-B-1);
(S)-4-(trifluoromethyl)-5-((1-(3-(4-(5-(trifluoromethyl)pyrimidin-2-yl)piperazine-1-carbonyl)-1H-pyrrol-1-yl)propan-2-yl)amino)pyridazin-3(2H)-one (I-C-1);
4-(trifluoromethyl)-5-((1-(1-(1-(5-(trifluoromethyl)pyrimidin-2-yl)piperidine-4-carbonyl)azetidin-3-yl)propan-2-yl)amino)pyridazin-3(2H)-one (I-D-1);
N-(4-((6-oxo-5-(trifluoromethyl)-1,6-dihydropyridazin-4-yl)amino)butyl)-4-(5-(trifluoromethyl)pyrimidin-2-yl)piperazine-1-carboxamide (I-E-1);
(E)-N-(4-((6-oxo-5-(trifluoromethyl)-1,6-dihydropyridazin-4-yl)amino)but-2-en-1-yl)-4-(5-(trifluoromethyl)pyrimidin-2-yl)piperazine-1-carboxamide (I-E-2);
N-(3-((6-oxo-5-(trifluoromethyl)-1,6-dihydropyridazin-4-yl)amino)propyl)-4-(2-(trifluoromethyl)pyrimidin-5-yl)piperazine-1-carboxamide (I-E-3);
N-(2-((6-oxo-5-(trifluoromethyl)-1,6-dihydropyridazin-4-yl)amino)ethyl)-4-(2-(trifluoromethyl)pyrimidin-5-yl)piperazine-1-carboxamide (I-E-4);
N-(3-((6-oxo-5-(trifluoromethyl)-1,6-dihydropyridazin-4-yl)amino)propyl)-4-(pyrimidin-5-yl)piperazine-1-carboxamide (I-E-5);
N-(2-((6-oxo-5-(trifluoromethyl)-1,6-dihydropyridazin-4-yl)amino)ethyl)-4-(pyrimidin-5-yl)piperazine-1-carboxamide (I-E-6); and
N-(3-(((6-oxo-5-(trifluoromethyl)-1,6-dihydropyridazin-4-yl)amino)methyl)phenyl)-4-(5-(trifluoromethyl)pyrimidin-2-yl)piperazine-1-carboxamide (I-E-7).

8. The pyridazinone compound according to claim 1, wherein the pharmaceutically acceptable salt is a salt formed by the pyridazinone compound and an acid, and the acid is hydrochloric acid, hydrobromic acid, sulfuric acid, phosphoric acid, carbonic acid, methanesulfonic acid, benzenesulfonic acid, p-toluenesulfonic acid, naphthalene sulfonic acid, citric acid, tartaric acid, lactic acid, pyruvic acid, acetic acid, maleic acid, succinic acid, fumaric acid, salicylic acid, phenylacetic acid, mandelic acid or ferulic acid.

9. A preparation method for the pyridazinone compound according to claim 2, wherein the preparation method is any of the following methods:
(1) when R^{t} is selected from trifluoromethyl, cyano or halogen, R² is hydrogen, R³ is selected from methyl or hydrogen, the carbon atom connected to R³ is the S configuration, or R² and R³ form a cyclopropyl, R⁴ is 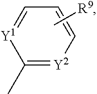
A¹ represents —NH—, A² is 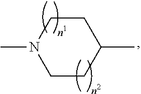
n¹ or n² is independently selected from 0 or 1, A³ is 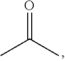
and A⁴ is 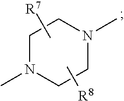
and a preparation method of a target compound I-A is as follows: 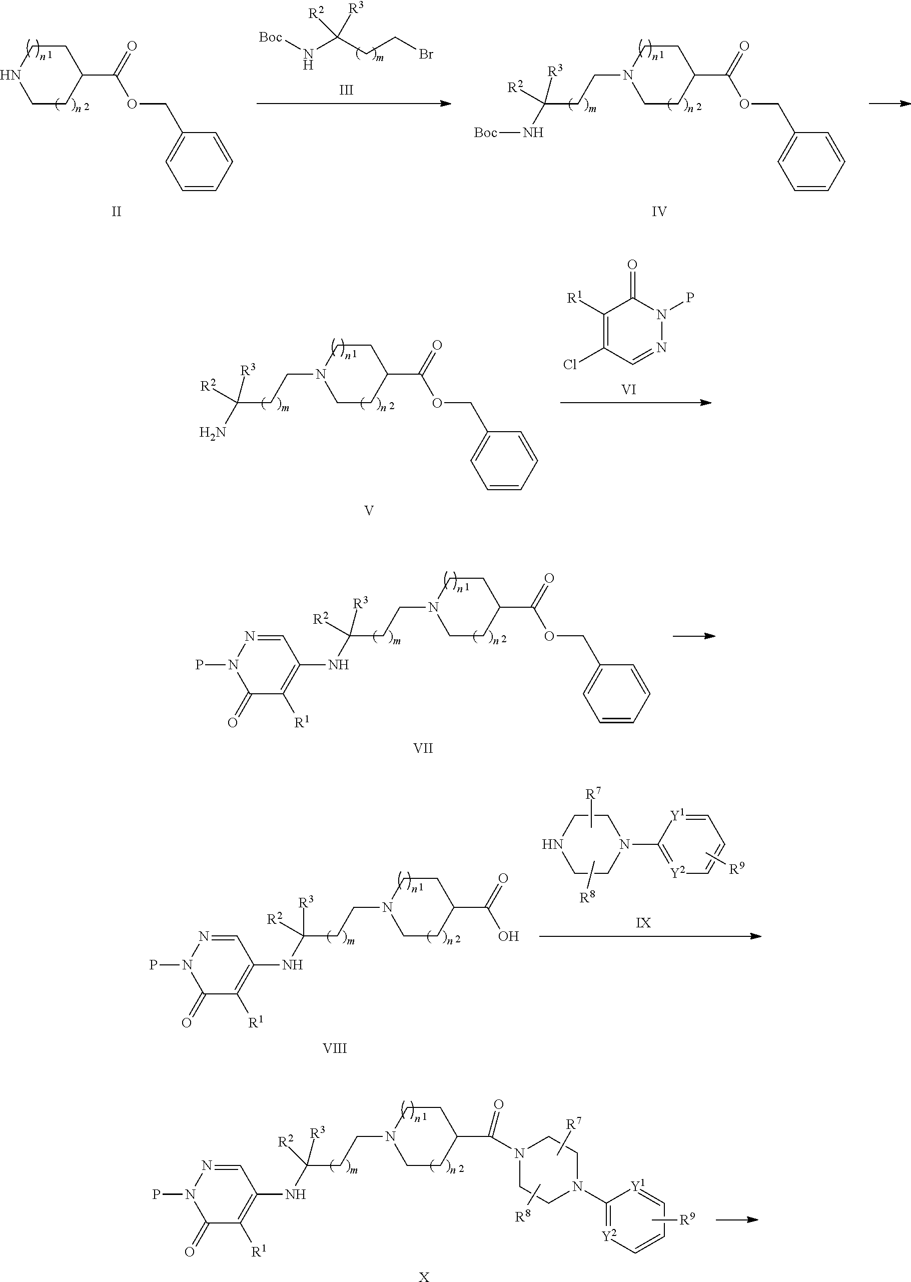 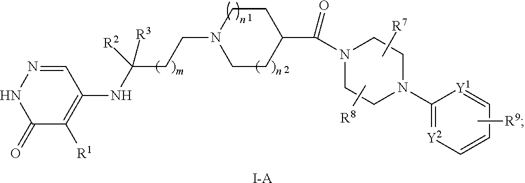
(2) when R^{t} is selected from trifluoromethyl, cyano or halogen, R² is hydrogen, R³ is selected from methyl or hydrogen, the carbon atom connected to R³ is the S configuration, or R² and R³ form a cyclopropyl, R⁴ is 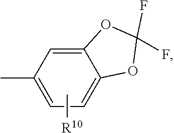
A¹ is —NH—, A² is 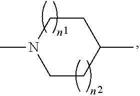
n¹ or n² is independently selected from 0 or 1, A³ is 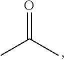
and A⁴ is 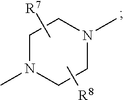
and a preparation method of a target compound I-B is as follows: 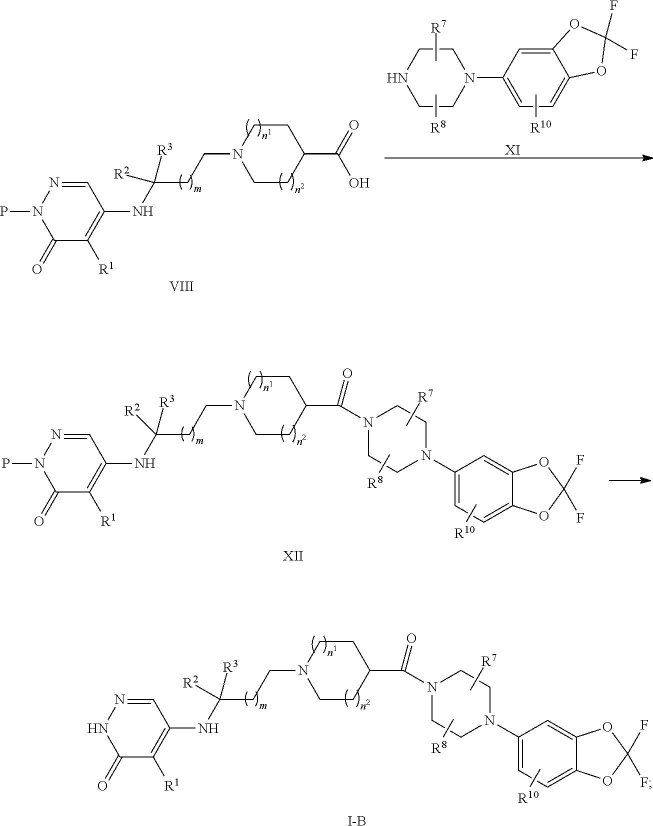
(3) when R¹ represents trifluoromethyl, cyano or halogen, R² is hydrogen, R³ is selected from methyl or hydrogen, the carbon atom connected to R³ is the S configuration, or R² and R³ form a cyclopropyl, R⁴ is 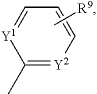
A¹ is —NH—, A² is 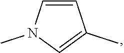
A³ is 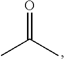
and A⁴ is 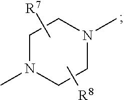
and a preparation method of a target compound I-C is as follows: 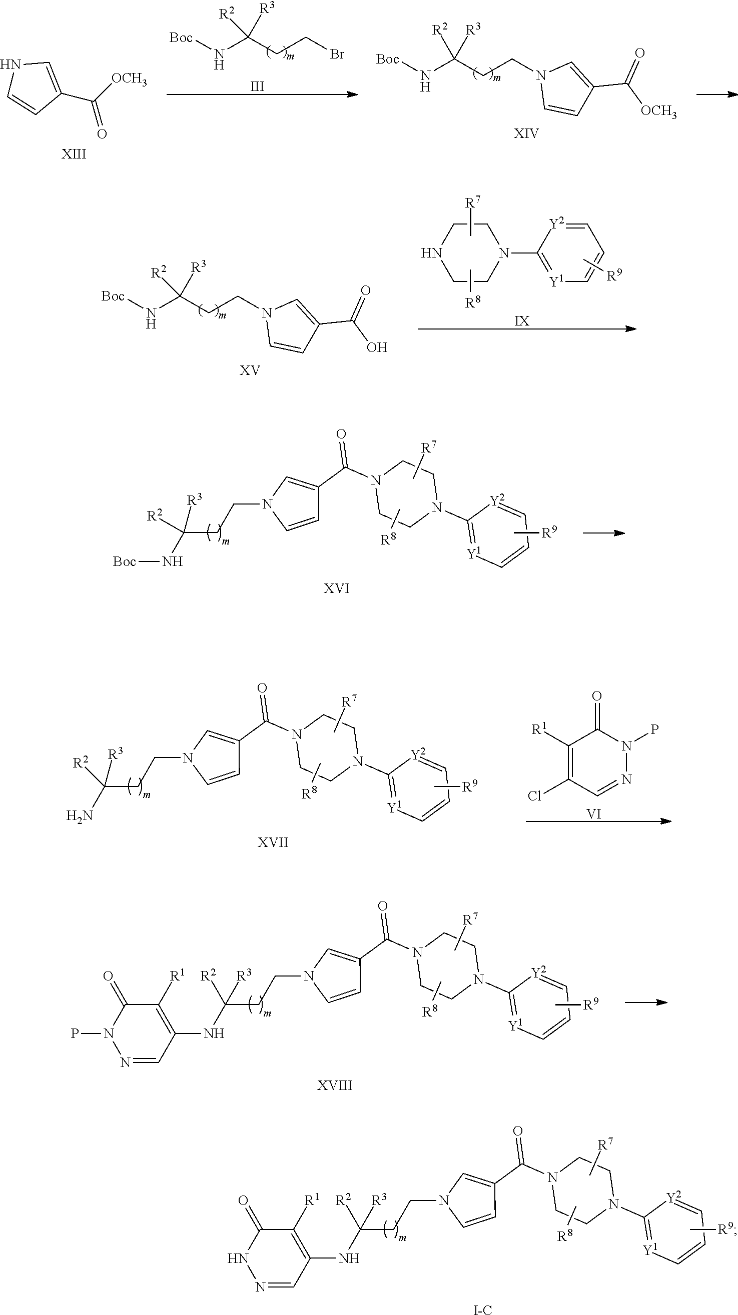
(4) when m=0, R¹ is selected from trifluoromethyl, cyano or halogen, R² is hydrogen, R³ is methyl, R⁴ is 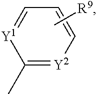
A¹ is —NH—, A² is 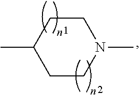
n¹ or n² is independently selected from 0 or 1, A³ is 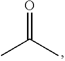
and A⁴ is 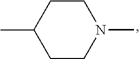
and a preparation method of a target compound I-D is as follows: 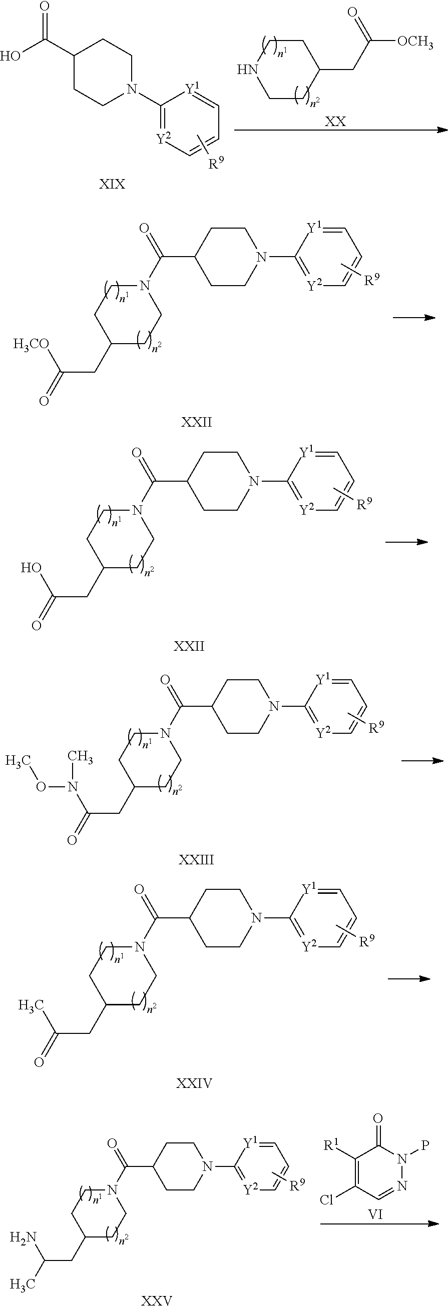 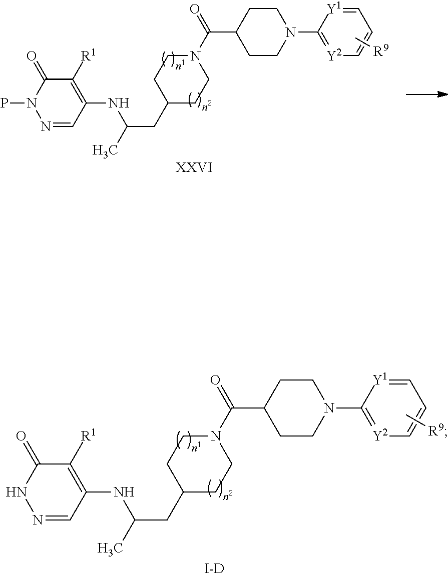
(5) when R¹ is selected from trifluoromethyl, cyano or halogen, R² and R³ are hydrogen, R⁴ is 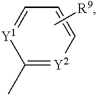
A¹ represents —NH—, A² is —NH— or —N(CH₃)—, A³ is 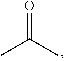
and A⁴ 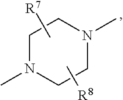
and a preparation method of a target compound I-E is as follows: 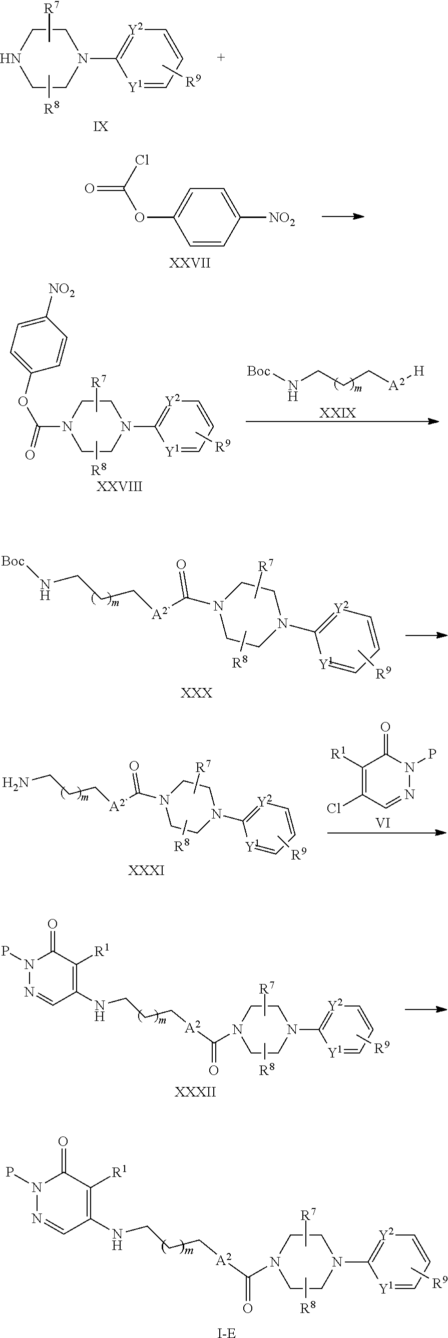
wherein m, Y¹, Y², R⁷, R⁸, R⁹ and R¹⁰ are defined as stated in claim 2; Boc is tert-butoxycarbonyl; P is (trimethylsilyl) ethoxymethyl or p-methoxybenzyl; and
the corresponding acid is prepared into a salt with Compound (I) prepared by the above method to obtain the pharmaceutically acceptable salt of the pyridazinone compound.

10. A pharmaceutical composition, wherein the pharmaceutical composition comprises the pyridazinone compound according to claim 1 and the pharmaceutically acceptable salt.

11. An application of the pyridazinone compound according to claim 1 in preparing a PARP7 inhibitor drug.

12. The application according to claim 11, wherein the drug is an anti-tumor drug.
